(19) United States
(12) Patent Application Publication
(10) Pub. No.: US 2024/0058343 A1
(43) Pub. Date: Feb. 22, 2024

(54) TREATMENT OF URTICARIA USING JAK INHIBITORS

(71) Applicant: Incyte Corporation, Wilmington, DE (US)

(72) Inventors: Cornelius P. Sailer, Leonia, NJ (US); Kurt Andrew Brown, Narberth, PA (US); Vijay Krishna Iyengar, Greenville, DE (US); James Lee, Devon, PA (US); Leandro Luiz Dos Santos, King of Prussia, PA (US); Susan Harless Smith, Pennsburg, PA (US)

(21) Appl. No.: 18/230,194

(22) Filed: Aug. 4, 2023

Related U.S. Application Data
(60) Provisional application No. 63/395,613, filed on Aug. 5, 2022.

(51) Int. Cl. A61K 31/519; A61K 9/00; A61K 31/4155; A61K 45/06; A61P 37/08

(52) U.S. Cl. CPC A61K 31/519; A61K 9/0053; A61K 31/4155; A61K 45/06; A61P 37/08

(57) ABSTRACT

The present application provides methods of treating urticaria in a patient in need thereof, comprising administering to the patient a therapeutically effective amount of a compound which inhibits JAK1, or a pharmaceutically acceptable salt thereof.

### PRIORITY CLAIM

This application claims the benefit of priority of U.S. Provisional Application No. 63/395,613, filed Aug. 5, 2022, which is incorporated herein by reference in its entirety.

### TECHNICAL FIELD

The present application provides methods for the treatment of urticaria using compounds that modulate the activity of Janus kinase (JAK) 1.

### BACKGROUND

Protein kinases (PKs) regulate diverse biological processes including cell growth, survival, differentiation, organ formation, morphogenesis, neovascularization, tissue repair, and regeneration, among others. Protein kinases also play specialized roles in a host of human diseases including cancer. Cytokines, low-molecular weight polypeptides or glycoproteins, regulate many pathways involved in the host inflammatory response to sepsis. Cytokines influence cell differentiation, proliferation and activation, and can modulate both pro-inflammatory and anti-inflammatory responses to allow the host to react appropriately to pathogens. Signaling of a wide range of cytokines involves the Janus kinase family (JAKs) of protein tyrosine kinases and Signal Transducers and Activators of Transcription (STATs). There are four known mammalian JAKs: JAK1 (Janus kinase-1), JAK2, JAK3 (also known as Janus kinase, leukocyte; JAKL; and L-JAK), and TYK2 (protein-tyrosine kinase 2).

Cytokine-stimulated immune and inflammatory responses contribute to pathogenesis of diseases: pathologies such as severe combined immunodeficiency (SCID) arise from suppression of the immune system, while a hyperactive or inappropriate immune/inflammatory response contributes to the pathology of autoimmune diseases (e.g., asthma, systemic lupus erythematosus, thyroiditis, myocarditis), and illnesses such as scleroderma and osteoarthritis (Ortmann, R. A., T. Cheng, et al. (2000) Arthritis Res 2(1): 16-32).

Deficiencies in expression of JAKs are associated with many disease states. For example, Jak1−/− mice are runted at birth, fail to nurse, and die perinatally (Rodig, S. J., M. A. Meraz, et al. (1998) Cell 93(3): 373-83). Jak2−/− mouse embryos are anemic and die around day 12.5 postcoitum due to the absence of definitive erythropoiesis.

The JAK/STAT pathway, and in particular all four JAKs, are believed to play a role in the pathogenesis of asthmatic response, chronic obstructive pulmonary disease, bronchitis, and other related inflammatory diseases of the lower respiratory tract. Multiple cytokines that signal through JAKs have been linked to inflammatory diseases/conditions of the upper respiratory tract, such as those affecting the nose and sinuses (e.g., rhinitis and sinusitis) whether classically allergic reactions or not. The JAK/STAT pathway has also been implicated in inflammatory diseases/conditions of the eye and chronic allergic responses.

Activation of JAK/STAT in cancers may occur by cytokine stimulation (e.g. IL-6 or GM-CSF) or by a reduction in the endogenous suppressors of JAK signaling such as SOCS (suppressor or cytokine signaling) or PIAS (protein inhibitor of activated STAT) (Boudny, V., and Kovarik, J., Neoplasm. 49:349-355, 2002). Activation of STAT signaling, as well as other pathways downstream of JAKs (e.g., Akt), has been correlated with poor prognosis in many cancer types (Bowman, T., et al. Oncogene 19:2474-2488, 2000). Elevated levels of circulating cytokines that signal through JAK/STAT play a causal role in cachexia and/or chronic fatigue. As such, JAK inhibition may be beneficial to cancer patients for reasons that extend beyond potential anti-tumor activity.

JAK2 tyrosine kinase can be beneficial for patients with myeloproliferative disorders, e.g., polycythemia vera (PV), essential thrombocythemia (ET), myeloid metaplasia with myelofibrosis (MMM) (Levin, et al., Cancer Cell, vol. 7, 2005: 387-397). Inhibition of the JAK2V617F kinase decreases proliferation of hematopoietic cells, suggesting JAK2 as a potential target for pharmacologic inhibition in patients with PV, ET, and MMM.

Inhibition of the JAKs may benefit patients suffering from skin immune disorders such as psoriasis, and skin sensitization. The maintenance of psoriasis is believed to depend on a number of inflammatory cytokines in addition to various chemokines and growth factors (JCI, 113:1664-1675), many of which signal through JAKs (Adv Pharmacol. 2000;47:113-74).

Urticaria is a heterogeneous group of diseases characterized by itchy hives and/or angioedema. Chronic spontaneous urticaria, formerly known as chronic idiopathic urticaria, is generally defined by the presence of wheals (hives), angioedema, or both for more than 6 weeks without an identifiable cause. The overall worldwide prevalence of CSU is approximately 1% with the potential for a high disease burden. Affected patients can experience an unpredictable disease course and duration with symptoms occurring in a spontaneous and recurrent manner and lasting over several years. Furthermore, severe pruritus and the sudden and unpredictable appearance of wheals and angioedema can impact sleep and patients' well-being.

Treatment of CSU remains challenging with nonsedating, second-generation, H1 antihistamines being first-line therapy at up to 4 times the recommended daily dose if needed. While second-generation, H1 antihistamines are effective in relieving symptoms for some patients, approximately 50% of individuals show insufficient response to high-dose, second-generation antihistamines. Omalizumab, an anti-IgE monoclonal antibody (administered subcutaneously), is recommended in combination with second-generation, H1 antihistamine treatment as a second line in the treatment algorithm, with only approximately 35% of patients achieving a complete response after 12 weeks. For those patients who continue to remain symptomatic with inadequate disease control, cyclosporin is recommended as a third-line treatment option in combination with second-generation, H1 antihistamines, although it is not licensed for urticaria and has a high incidence of adverse effects, limiting its use in many patients with CSU. Thus, there is a need for other second- and third-line treatment options that can be administered orally and potentially offer an improved efficacy and side-effect profile for the management of CSU.

Thus, new or improved agents which inhibit kinases such as JAKs are continually needed for developing new and more effective pharmaceuticals that are aimed at augmentation or suppression of the immune and inflammatory pathways, such as the treatment of urticaria. This application is directed to that need and others.

### DESCRIPTION OF THE DRAWINGS

FIG. 1 depicts a graphical representation of Compound 1 mediated pharmacological inhibition of lesional skin genes using JAK1 inhibitor Compound 1.

FIG. 2 depicts an outline of a phase 2 randomized, double-blind, placebo-controlled dose-ranging study of the efficacy and safety of Compound 1.

### SUMMARY

The present application provides methods of treating urticaria in a patient in need thereof, comprising administering to the patient a therapeutically effective amount of a compound which inhibits JAK1, or a pharmaceutically acceptable salt thereof.

In some embodiments, the compound or salt is selective for JAK1 over JAK2, JAK3, and TYK2.

In some embodiments, the compound is {1-{1-[3-fluoro-2-(trifluoromethyl)isonicotinoyl]piperidin-4-yl}-3-(7H-pyrrolo[2,3 -d]pyrimidin-4-yl)-1H-pyrazol-1-yl}azetidin-3-yl}acetonitrile, or a pharmaceutically acceptable salt thereof.

In some embodiments, the salt is {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile adipic acid salt.

In some embodiments, the compound is 4-[3-(cyanomethyl)-3-(3′,5′-dimethyl-1H, 1′H-4,4′-bipyrazol-1-yl)azetidin-1-yl]-2,5-difluoro-N-[(1S)-2,2,2-trifluoro-1-methylethyl]benzamide (Compound 1), or a pharmaceutically acceptable salt thereof.

In some embodiments, the salt is 4-[3-(cyanomethyl)-3-(3′,5′-dimethyl-1H, 1′H-4,4′-bipyrazol-1-yl)azetidin-1-yl]-2,5- difluoro-N-[(1S)-2,2,2-trifluoro-1-methylethyl]benzamide phosphoric acid salt.

In some embodiments, the compound or salt is administered at a dosage of 15, 30, 45 or 75 mg on a free base basis.

In some embodiments, the compound is 42R,5S)-5-{2-[(1R)-1-hydroxyethyl]-1H-imidazo[4,5-d]thieno[3,2-b]pyridin-1-yl}tetrahydro-2H-pyran-2-yl)acetonitrile, or a pharmaceutically acceptable salt thereof.

In some embodiments, the compound is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile monohydrate.

In some embodiments, the methods further comprise administering an additional therapeutic agent (e.g., an antibiotic, a retinoid, a corticosteroid, an anti-TNF-alpha agent, or an immunosuppressant).

In some embodiments, the methods further comprise administering an additional therapeutic agent, where the additional therapeutic agent is an antihistamine, and where the antihistamine is a second-generation H1 antihistamine.

In some embodiments, the administrating of the compound or salt is topical. In some embodiments, the administering of the compound or salt is oral.

In some embodiments, the method results in about a 10% to about a 90% improvement in a number and/or size of welts.

In some embodiments, the method results in about a 10%, 20%, 30%, 40%, or 50% improvement in a number and/or size of welts.

In some embodiments, the method results in about a 10% to about a 90% improvement in a severity of hives (e.g., based on HSS7 (hive severity score over 7 days)).

In some embodiments, the method results in about a 10%, 20%, 30%, 40%, or 50% improvement in a severity of hives (e.g., based on HSS7).

In some embodiments, the method results in about a 10% to about a 90% improvement in a severity of angioedema (e.g., based on AAS7 (angioedema activity score over 7 days)).

In some embodiments, the method results in about a 10%, 20%, 30%, 40%, or 50% improvement in a severity of angioedema (e.g., based on AAS7).

In some embodiments, the method results in about a 10% to about a 90% improvement from baseline in itch severity score (ISS) or weekly itch severity score over 7 days (ISS7).

In some embodiments, the method results in about a 10%, 20%, 30%, 40%, or 50% improvement from baseline in ISS or ISS7.

In some embodiments, the method results in about a 10% to about a 90% improvement from baseline in urticaria activity score (UAS) or urticaria activity score over 7 days (UAS7).

In some embodiments, the method results in about a 10%, 20%, 30%, 40%, or 50% improvement from baseline in UAS or UAS7.

In some embodiments, the method results in about a 10% to about 90% improvement from baseline in weekly urticaria control test (UCT).

In some embodiments, the method results in about a 10%, 20%, 30%, 40%, or 50% improvement from baseline in weekly urticaria control test (UCT).

In some embodiments, the method results in about a 10% to about a 90% improvement in quality of life and/or other Patient Reported Outcomes (PROs).

In some embodiments, the method results in about a 10%, 20, 30%, 40%, or 50% improvement in quality of life and/or other PROs.

The present application also provides a compound which inhibits JAK1, or a pharmaceutically acceptable salt thereof, for use in treating urticaria, with or without angioedema and the types of urticaria including cholinergic urtiaria, cold induced urticaria (CINDU), dermatographism/urticaria factitism, heat urticaria, delayed pressure urticaria, solar urticaria, contact urticaria, and aquagenic urticaria.

The present application also provides a compound which inhibits JAK1, or a pharmaceutically acceptable salt thereof, for use in treating urticaria.

The present application further provides use of a compound which inhibits JAK1, or a pharmaceutically acceptable salt thereof, for preparation of a medicament for use in the treatment of urticaria.

### DETAILED DESCRIPTION

The present application provides, inter alia, a method of treating urticaria in a patient in need thereof, comprising administering a therapeutically effective amount of compound which inhibits JAK1, or a pharmaceutically acceptable salt thereof.

The method described herein utilize compound or salts that are inhibitors of JAK1. In some embodiments, the compound is:

{1-{1-[3-Fluoro-2-(trifluoromethyl)isonicotinoyl]piperidin-4-yl}-3-[4-(7H-pyrrolo[2,3 -d]pyrimidin-4-yl)-1H- pyrazol-1-yl]azetidin-3-yl}acetonitrile;

4-{3-(Cyanomethyl)-3-[4-(7H-pyrrolo[2,3-d]pyrimidin-4-yl)-1H-pyrazol-1-yl]azetidin-1-yl}-N-[4-fluoro-2-(trifluoromethyl)phenyl]piperidine-1-carboxamide;

[3-[4-(7H-pyrrolo[2,3 -d]pyrimidin-4-yl)-1H-pyrazol-1-yl]-1-{[2-(trifluoromethyl)pyrimidin-4-yl]carbonyl}piperidin-4-yl)azetidin-3-yl]acetonitrile;

4-[3-(cyanomethyl)-3-(3′,5′-dimethyl-1H-4,4′-bipyrazol-1-yl)azetidin-1-yl]-2,5-difluoro-N-[(1S)-2,2,2-1 S)-2,2,2-trifluoro-1-methylethyl]benzamide;

((2R,5 S)-5-{2-[(1R)-1-hydroxyethyl]-1H-imidazo[4,5-d]thieno[3,2-b]pyridin-1-yl}tetrahydro-2H-pyran-2-yl)acetonitrile;

3-[1-(6-chloropyridin-2-yl)pyrrolidin-3-yl]-3-(7H-pyrrolo[2,3-d]pyrimidin-4-yl)-1H-pyrazol-1-yl]propanenitrile;

3-(1[1,3]oxazolo[5,4-b]pyridin-2-ylpyrrolidin-3-yl)-3-[4-(7H-pyrrolo[2,3-d]pyrimidin-4-yl)-1H-pyrazol-1-yl]propanenitrile;

4-[(4-{3-cyano-2-[4-(7H-pyrrolo[2,3-d]pyrimidin-4-yl)-1H-pyrazol-1-yl]propyl}piperazin-1-yl)carbonyl]-3-fluorobenzonitrile;

4-[(4-{3-cyano-2[3-(7H-pyrrolo[2,3-d]pyrimidin-4-yl)-1H-pyrrol-1-yl]propyl}piperazin-1-yl)carbonyl]-3-fluorobenzonitrile;

[trans-1-[4-(7H-pyrrolo[2,3 -d]pyrimidin-4-yl)-1H-pyrazol-1-yl]-3-(4-{[2-(trifluoromethyl)pyrimidin-4-yl]carbonyl}piperazin-1-yl)cyclobutyl]acetonitrile;

{trans-3-(4-{[4-[(3 -hydroxyazetidin-1-yl)methyl]-6-(trifluoromethyl)pyridin-2-yl]oxy}piperidin-1-yl)-1-[4-(7H-pyrrolo[2,3-d]pyrimidin-4-yl)-1H-pyrazol-1-yl] cycl obutyl}acetonitrile;

{trans-3-(4-{[4-{[(2S)-2-(hydroxymethyl)pyrrolidin-1-yl]methyl}1-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;

{trans-3-(4-{[4-{[(2R)-2-(hydroxymethyl)pyrrolidin-1-yl]methyl}-6-(trifluoromethyl)pyridin-2-yl]oxy}piperidin-1-yl)-1-[4-(7H-pyrrolo[2,3 -d]pyrimidin-4-yl)-1H-pyrazol-1-yl]cyclobutyl}acetonitrile;

4-(4-{3-[(dimethylamino)methyl]-5-fluorophenoxy}piperidin-1-yl)-3-[4-(7H-pyrrolo[2,3-d]pyrimidin-4-yl)-1H- pyrazol-1-yl]butanenitrile;

5-{3-(cyanomethyl)-3-[4-(7H-pyrrolo[2,3-d]pyrimidin-4-yl)-1H-pyrazol-1-yl]azetidin-1-yl}-N-isopropylpyrazine-2-carboxamide;

4-3-(cyanomethyl)-3-[4-(7H-pyrrolo[2,3-d]pyrimidin-4-yl)-1H-pyrazol-1-yl]azetidin-1-yl -2,5-difluoro-N-[(1S)-2,2,2-trifluoro-1-methylethyl]benzamide;

5-5 3-(cyanomethyl)-3-[4-(1H-pyrrolo[2,3-b]pyridin-4-yl)-1H-pyrazol-1-yl]azetidin-1-yl-N-isopropylpyrazine-2-carboxamide;

{1-(cis-4-{[6-(2-hydroxyethyl)-2-(trifluoromethyl)pyrimidin-4-yl]oxy}cyclohexyl)-3-[4-(7H-pyrrolo[2,3-d]pyrimidin-4-yl)-1H-pyrazol-1-yl]azetidin-3-yl}acetonitrile;

{1-(cis-4-{[4-[(ethylamino)methyl]-6-(trifluoromethyl)pyridin-2-yl]oxycyclohexyl)-3-[4-(7H-pyrrolo[2,3-d]pyrimidin-4-yl)-1H-pyrazol-1-yl]azetidin-3-yl}acetonitrile;

{1-(cis-4-{[4-(1-hydroxy-1-methylethyl)-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;

{1-(cis-4-{[4-{[(3S)-3-hydroxypyrrolidin-1-yl]methyl}-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;

{1-(cis-4-{[4-{[(3S)-3-hydroxypyrrolidin-1-yl]methyl}-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl acetonitrile;

{trans-3-(4-1[4-({[(1S)-2-hydroxy-1-methylethyl]amino}methyl)-6-(trifluoromethyppyridin-2-yl]oxy}piperidin-1-yl)-1-[4-(7H-pyrrolo[2,3-d]pyrimidin-4-yl)-1H-pyrazol-1-yl]cyclobutyl}acetonitrile;

{trans-3-(4-{8 4-({[(2R)-2-hydroxypropyl]amino}methyl)-6-(trifluoromethyppyridin-2-yl]oxy}piperidin-1-yl)-1-[4-(7H-pyrrolo[2,3-d]pyrimidin-4-yl)-1H-pyrazol-1-yl]cyclobutyl}acetonitrile{trans-3-(4-{[4-({[(2S)-2-hydrox;

ypropyl]amino}methyl)-6-(trifluoromethyl)pyridin-2-yl]oxy}piperidin-1-yl)-1-[4-(7H-pyrrolo[2,3-d]pyrimidin-4-yl)-1H-pyrazol-1-yl]cycobuty}acetonitrile;

{trans-3-(4-{[4-(2-hydroxyethyl)-6-(trifluoromethyppyridin-2-yl]oxy}piperidin-1-yl)-[4-(7H-pyrrolo[2,3 -d]pyrimidin-4-yl)-1H-pyrazol-1-yl]cyclobutyl}acetonitrile;

or a pharmaceutically acceptable salt of any of the aforementioned.

In some embodiments, the compound or salt is a JAK1 inhibitor. In some embodiments, the compound or salt is selective for JAK1 over JAK2, JAK3 and TYK2. For example, some of the compounds described herein, or a pharmaceutically acceptable salt thereof, preferentially inhibit JAK1 over one or more of JAK2, JAK3, and TYK2. JAK1 plays a central role in a number of cytokine and growth factor signaling pathways that, when dysregulated, can result in or contribute to disease states. For example, IL-6 levels are elevated in rheumatoid arthritis, a disease in which it has been suggested to have detrimental effects (Fonesca, et al., Autoimmunity Reviews, 8:538-42, 2009). Because IL-6 signals, at least in part, through JAK1, IL-6 can be indirectly through JAK1 inhibition, resulting in potential clinical benefit (Guschin, et al. Embo J 14:1421, 1995; Smolen, et al. Lancet 371:987, 2008). Moreover, in some cancers JAK1 is mutated resulting in constitutive undesirable tumor cell growth and survival (Mullighan, Proc Natl Acad Sci USA. 106:9414-8, 2009; Flex, J Exp Med. 205:751-8, 2008). In other autoimmune diseases and cancers, elevated systemic levels of inflammatory cytokines that activate JAK1 may also contribute to the disease and/or associated symptoms. Therefore, patients with such diseases may benefit from JAK1 inhibition. Selective inhibitors of JAK1 may be efficacious while avoiding unnecessary and potentially undesirable effects of inhibiting other JAK kinases.

Urticaria (also more commonly referred to as hives) is characterized by significant skin inflammation. Urticaria can be characterized by welts that vary in size and shape. Urticaria can be characterized by itching that can be severe. Urticaria can be characterized by angioedema (painful swelling of the lips, eyelids, and/or inside the throat). Presented herein are Examples that support the hypothesis that the inflammation is driven, in large part, by JAK/STAT mediated pathways. Therefore, patients with urticaria may benefit from JAK1 inhibition. Selective inhibitors of JAK1 may be efficacious while avoiding unnecessary and potentially undesirable effects of inhibiting other JAK kinases.

Urticaria is an autoimmune, mast-cell driven disease, presenting with chronic itch and characterized by spontaneous and recurrent appearance of wheals, angioedema, or both for >6 consecutive weeks. Urticaria has no known initial trigger. Urticaria lesions exhibit strong T cell infiltrate. IL-6 expression is upregulated in Urticaria lesions. Expression of IL-33, IL-25, and TSLP (from epithelial cells) trigger Mast Cell (MC). Autoimmune Ab (IgE or IgG) activate MC. Activated MC secrete vasoactive mediators, cytokines and chemokines that promote immune infiltration. Increased serum levels of Th1, Th2 and Th17-related cytokines) correlate with the disease severity of urticaria. JAK1/2 inhibition abrogates cytokine signaling from IL-6 and Th1/Th2/Th22-related cytokines. JAK1/2 inhibition can modulate MC activation, including degranulation and cytokine production. JAK1/2 inhibition may interfere with itch signaling on cutaneous nerve endings (i.e., TSLP, IL31, IL13 receptor signaling). JAK1/2 inhibition reduces chemokine secretion, leading to less cellular infiltrate (i.e., CXCL10).

The overall prevalence of urticaria is ˜0.7% of the world population. Specifically the prevalence is ˜0.7% in North America, ˜0.5% in Europe, ˜1.5% in Latin America, and ˜1.4% in Asia. Onset of urticaria is typically 20 s-40 s. Women typically suffer from urticaria twice as much as men and 70% of people who suffer are Caucasian.

Current typical treatments include standard or high dose 2 ^{nd} generation antihistamines (1^{st} line and 2^{nd}line), although ˜50% of patients show insufficient response to high-dose antihistamines. The only drug approved is omalizumab (anti-IgE, 3^{rd} line, add-on therapy), although ˜35% of patients achieve complete response with omalizumab. Ornalizurnab is a humanized anti-IgE inonoclornal antibody that binds and captures circulating IgE which prevents interaction with receptors on mast cells and basophils, thereby interrupting the allergic cascade. Problems can arise with Omalizumab in, for example, with patients with low IgE levels that high doses of anti IgE will not bind. As such, blocking other pathways with a JAK1 inhibitor that reduce, for example, IgE, Il-4, Il-13, and TSLP, leads to greater efficacy for a broader range of patients (especially patients who failed to received satisfactory treatment using antihistamines such Omalizurnab). In some embodiments, JAK1 inhibitors are used for treating patients with CSU that is inadequately controlled by second-generation Hi antihistamines.

In some embodiments, urticaria includes subtypes spontaneous urticaria and physical urticaria. In some embodiments, spontaneous urticaria includes acute urticaria and chronic urticaria (including but not limited to chronic continuous urticaria and chronic recurrent urticaria). In some embodiments, physical urticaria includes dermographic urticaria, delayed pressure urticaria, cold contact urticaria, heat contact urticaria, solar urticaria, and vibratory urticaria/angioedema. In some embodiments, urticaria includes subtypes which have been referred to as special types of urticaria including cholinergic urticaria, adrenergic urticaria, contact urticaria (allergic or pseudoallergic), and aquagenic urticaria. Examples of diseases related to urticaria historically include urticaria pigmentosa (mastocytosis), urticarial vasculitis, and familial cold urticaria.

Target population includes patients with Chronic Spontaneous Urticaria (CSU), defined as the presence of recurrent urticaria (hives or wheals), angioedema, or both, for greater than 6 weeks or longer with symptoms at least three to four times per week, with no known trigger and who have failed to respond to 4x the daily dose of second-generation antihistamines.

In Europe Chronic Spontaneous Urticaria (CSU) is defined as the presence of recurrent urticaria (hives or wheals), angioedema, or both, for greater than 6 weeks or longer with symptoms at least three to four times per week, with no known trigger and who have inadequate response to, are contraindicated to or intolerant to previous biological therapy.

In some embodiments, use is for chronic spontaneous urticaria (CSU) in adults and adolescents 12 years of age and older who remain symptomatic despite H1 antihistamine treatment. Dosage can include 15 mg, 30 mg, 45 mg, and/or 75 mg. Dosage can include 15 or 30 mg of a JAK1 inhibitor. Dosage can include 15 mg of a JAK1 inhibitor. Dosage can include 30 mg of a JAK1 inhibitor.

In some embodiments, an endpoint (e.g., primary) can include a change from baseline in weekly itch severity score (except in certain countries/regions (e.g., EU and EU reference countries)) [e.g., Time Frame: Baseline to Week 24]. A change from baseline in weekly itch severity score (ISS7) at e.g., Week 24 can include at least 25% or at least 30% PBO adjusted. A change from baseline in weekly urticaria activity score (UAS7, composite patient reported itch and hive score) at e.g., Week 24 can include at least 25% or at least 30% PBO adjusted.

In some embodiments, a change in baseline in the UAS7 is defined as the 7-day sum of the individual, daily recorded scores for HSS and ISS, for example at week 12.

In some embodiments, the method results in about a 10% to about a 90% improvement in a severity of hives (e.g., based on HSS or HSS7 (hive severity score over 7 days)).

In some embodiments, the method results in about a 10%, 20%, 30%, 40%, or 50% improvement in a severity of hives (e.g., based on HSS or HSS7).

In some embodiments, the method results in about a 10% to about a 90% improvement in a severity of angioedema (e.g., based on AAS or AAS7 (angioedema activity score over 7 days)). In some embodiments, the method results in about a 10%, 20%, 30%, 40%, or 50% improvement in a severity of angioedema (e.g., based on AAS or AAS7). The AAS records if participants have experienced a swelling episode in the previous 24 hours. If the participant responds yes, further questions are asked covering time of event, physical discomfort, impact on daily activities, appearance, and overall severity. If a participant has entered at least 5 daily AAS scores within the 7 days prior to the study visit, the AAS7 score is calculated as the sum of the available AAS scores, divided by the number of days that have an AAS score, multiplied by 7. In some embodiments, endpoints (e.g., secondary) can include a change from baseline in weekly urticaria activity score (UAS or UAS7) at e.g., Week 12 and e.g., Week 24 of at least 25% or at least 30% PBO adjusted at e.g., 24 weeks. A change from baseline in ISS7 (EU) can include at least 25% or at least 30% PBO Adjusted at eg., week 24. The UAS is a composite score with numeric severity intensity ratings (0=none to 3=intense/severe) for a) the number of hives (i.e., HSS) and b) the intensity of the pruritus (i.e., ISS) over the past 24 hours. The UAS7 is the 7-day sum of the daily UAS. The UAS7 (range 0 to 42) is equal to the ISS7 (range 0 to 21) plus the HSS7 (range 0 to 21). If a participant has entered at least 5 daily UAS scores within the 7 days prior to the study visit, the UAS7 score is calculated as the sum of the available UAS scores, divided by the number of days that have a UAS score, multiplied by 7. If there are more than 2 daily UAS scores missing within the prior 7 days, then the UAS7 score is missing for the week.

In some embodiments, the method results in about a 10% to about 90% improvement from baseline in weekly urticaria control test (UCT). UCT is a way to measure urticaria disease activity. An example scale for UCT is 0-16 where 0 equates to most severe activity and 16 equates to no disease activity. In some embodiments, a score of less than 12 on the UCT identifies subjects with poorly controlled chronic urticaria, and a score of greater than or equal to 12 identifies subjects with well-controlled symptoms. In some embodiments, an improvement in 3 points is a minimal response, and an improvement of greater than or equal to 6 points is a marked response.

In some embodiments, the method results in about a 10%, 20%, 30%, 40%, or 50% improvement from baseline in weekly urticaria control test (UCT).

In some embodiments, the method results in about a 10% to about a 90% improvement in quality of life and/or other Patient Reported Outcomes (PROs). In some embodiments, the method results in about a 10%, 20%, 30%, 40%, or 50% improvement in quality of life and/or other PROs. In some embodiments, efficacy of the treatment method disclosed herein can be established based upon patient-reported outcomes (PROs). In some embodiments, efficacy of the treatment method disclosed herein can be established based upon a Dermatology Life Quality Index (DLQI). In some embodiments, Compound 1 and/or methods of use described herein result in an improvement in a participant's response to DLQI from baseline. The DLQI is a validated questionnaire (e.g., 10-question) to measure how much the skin problem has affected the participant over the previous 7 days. The participant will answer the questionnaire with either (1) very much, (2) a lot, (3) a little, or (4) not at all. The questionnaire can be analyzed under 6 headings: symptoms and feelings; daily activities; leisure; work and school; personal relations; and treatment.

In some embodiments, the method results in about a 10% to about a 90% improvement in Chronic Urticaria Quality of Life Questionnaire (CU-Q2oL). In some embodiments, the method results in about a 10%, 20%, 30%, 40%, or 50% improvement in CU-Q2oL. In some embodiments, efficacy of the treatment method disclosed herein can be established based upon CU-Q2oL. Participants will complete a CU-Q2oL validated questionnaire at the study visits. The CU-Q2oL can be a 23-item, CSU-specific, health-related, quality-of-life questionnaire. Participants rate their CSU symptoms and the impact of their CSU on various aspects of their lives over the previous 14 days. An overall score can be calculated as well for the following domains: pruritus, swelling, impact on life activities, sleep problems, limits, and looks.

In some embodiments, the method results in about a 10% to about a 90% improvement in Angioedema Quality of Life Questionnaire (AE-QoL). In some embodiments, the method results in about a 10%, 20%, 30%, 40%, or 50% improvement in AE-QoL. Participants will complete the AE-QoL validated questionnaire at study visits. The AE-QoL is a validated tool used as a measure of quality-of-life impairment due to angioedema. The AE-QoL consists of 17 questions covering the following 4 domains: functioning, fatigue/mood, fears/shame, and food. Participants will answer how often in the last 4 weeks each item was affected due to swelling episodes.

In some embodiments, the method results in about a 10% to about a 90% improvement in Work Productivity and Activity Index—Chronic Urticaria (WPAI-CU). In some embodiments, the method results in about a 10%, 20%, 30%, 40%, or 50% improvement in WPAI-CU. Participants will complete a WPAI-CU questionnaire at the study visits. The WPAI-CU questionnaire is a 6-item, validated instrument designed to measure impairments in both paid and unpaid work. It measures absenteeism and presenteeism as well as impairments in unpaid activity because of health problems during the past 7 days. The minimum clinically important difference is defined as a one-half STD of the total population's baseline score. Absenteeism, presenteeism, and overall work impairment will be assessed only for employed participants.

In some embodiments, the method results in about a 10% to about a 90% improvement in EuroQol 5-Dimension 5-Level Scale (EQ-5D-5L). In some embodiments, the method results in about a 10%, 20%, 30%, 40%, or 50% improvement in EQ-5D-5L. Participants will complete the EQ-5D-5L questionnaire. The EQ-5D-5L is a standardized instrument for use as a measure of health outcomes. The EQ-5D-5L will provide data for use in economic models and analyses, including developing health utilities or quality-adjusted life-years. The EQ-5D-5L consists of 2 sections: the EQ-5D descriptive system and the EQ VAS, which asks about the participant's health for that day. The descriptive system comprises 5 dimensions: mobility, self-care, usual activities, pain/discomfort, and anxiety/depression. Each dimension has 5 levels: no problems, slight problems, moderate problems, severe problems, and extreme problems. The EQ VAS records the participant's self-rated health on a vertical VAS (0 to 100), on which the anchors are labeled as “The best health you can imagine” and “The worst health you can imagine.”

In some embodiments, the method results in about a 10% to about a 90% improvement in Patient Global Impression of Change (PGI-C). In some embodiments, the method results in about a 10%, 20%, 30%, 40%, or 50% improvement in PGI-C. The PGI-C questionnaire will be completed according to a schedule. The PGI-C instrument will provide data on the overall response to treatment from the participant's perspective. The PGI-C is a single-item questionnaire about the degree of change in the participant's overall CSU status compared with the start of treatment, using a 7-point categorical response scale ranging from 1 (very much improved) to 7 (very much worse).

In some embodiments, the method results in about a 10% to about a 90% improvement in Patient Global Impression of Severity (PGI-S). In some embodiments, the method results in about a 10%, 20%, 30%, 40%, or 50% improvement in PGI-S. Participants will complete the PGI-S questionnaire. The PGI-S will provide data on CSU symptom severity from the participant's perspective. The PGI-S is a single-item questionnaire to evaluate disease severity. Participants will rate their CSU symptoms experienced at each study visit using a 5-point scale (none, mild, moderate, severe, very severe).

In some embodiments, the compound or salt inhibits JAK1 preferentially over JAK2 (e.g., have a JAK2/JAK1 IC₅₀ ratio >1). In some embodiments, the compounds or salts are about 10-fold more selective for JAK1 over JAK2. In some embodiments, the compounds or salts are about 3-fold, about 5-fold, about 10-fold, about 15-fold, or about 20-fold more selective for JAK1 over JAK2 as calculated by measuring IC₅₀ at 1 mM ATP (see Example A).

In some embodiments, the JAK1 inhibitor is a compound of Table 1, or a pharmaceutically acceptable salt thereof. The compounds in Table 1 are selective JAK1 inhibitors (selective over JAK2, JAK3, and TYK2). The IC₅₀ values obtained by the method of Example A at 1 mM ATP are shown in Table 1.

TABLE 1

| | | | | JAK1 | |
|---|---|---|---|---|---|
| Comp. | | | | IC₅₀ | JAK2/ |
| No. | Prep. | Name | Structure | (nM) | JAK1 |
| 1 | US 2011/ 0224190 (Example 1) | {1-{1-[3-Fluoro-2- (trifluoromethyl)isonico- tinoyl]piperidin-4-yl}-3- [4-(7H-pyrrolo[2,3- d]pyrimidin-4-yl)-1H- pyrazol-1-yl]azetidin-3- yl}acetonitrile | 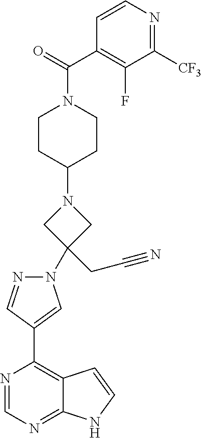 [SMILES: N#CCC1(n2cc(-c3ncnc4[nH]ccc34)cn2)CN(C2CCN(C(=O)c3ccnc(C(F)(F)F)c3F)CC2)C1] | + | >10 |
| 2 | US 2011/ 0224190 (Example 154) | 4-{3-(Cyanomethyl)-3- [4-(7H-pyrrolo[2,3- d]pyrimidin-4-yl)-1H- pyrazol-1-yl]azetidin-1- yl}-N-[4-fluoro-2- (trifluoromethyl)phenyl] piperidine-1- carboxamide | 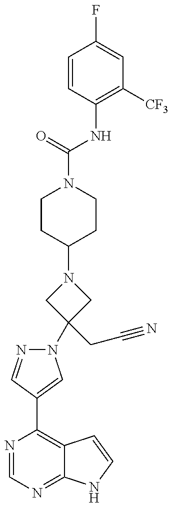 [SMILES: N#CCC1(n2cc(-c3ncnc4[nH]ccc34)cn2)CN(C2CCN(C(=O)Nc3ccc(F)cc3C(F)(F)F)CC2)C1] | + | >10 |
| 3 | US 2011/ 0224190 (Example 85) | [3-[4-(7H-pyrrolo[2,3- d]pyrimidin-4-yl)-1H- pyrazol-1-yl]-1-(1-{[2- (trifluoromethyl)pyrimi- din-4- yl]carbonyl}piperidin-4- yl)azetidin-3- yl]acetonitrile | 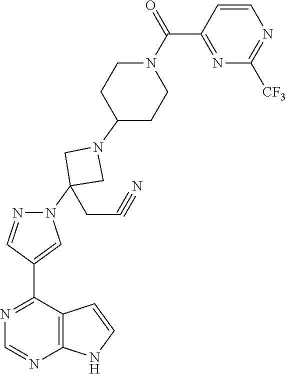 [SMILES: N#CCC1(n2cc(-c3ncnc4[nH]ccc34)cn2)CN(C2CCN(C(=O)c3ccnc(C(F)(F)F)n3)CC2)C1] | + | >10 |
| 4 | US 2014/ 0343030 (Example 7) | 4-[3-(cyanomethyl)-3- (3′,5′-dimethyl-1H,1′H- 4,4′-bipyrazol-1- yl)azetidin-1-yl]-2,5- difluoro-N-[(1S)-2,2,2- trifluoro-1- methylethyl]benzamide | 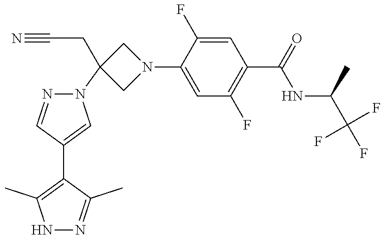 [SMILES: Cc1n[nH]c(C)c1-c1cnn(C2(CC#N)CN(c3cc(F)c(C(=O)N[C@@H](C)C(F)(F)F)cc3F)C2)c1] | +++ | >10 |
| 5 | US 2014/ 0121198 (Example 20) | ((2R,5S)-5-{2-[(1R)-1- hydroxyethyl]-1H- imidazo[4,5- d]thieno[3,2-b]pyridin- 1-yl}tetrahydro-2H- pyran-2-yl)acetonitrile | 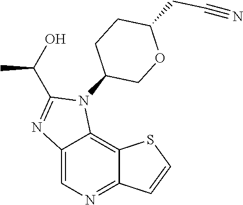 [SMILES: C[C@@H](O)c1nc2cnc3ccsc3c2n1[C@H]1CC[C@H](CC#N)OC1] | ++ | >10 |
| 6 | US 2010/ 0298334 (Example 2)^{a} | 3-[1-(6-chloropyridin-2- yl)pyrrolidin-3-yl]-3-[4- (7H-pyrrolo[2,3- d]pyrimidin-4-yl)-1H- pyrazol-1- yl]propanenitrile | 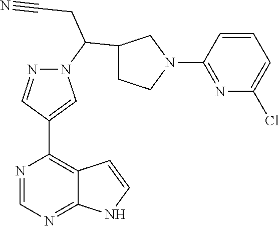 [SMILES: N#CCC(C1CCN(c2cccc(Cl)n2)C1)n1cc(-c2ncnc3[nH]ccc23)cn1] | + | >10 |
| 7 | US 2010/ 0298334 (Example 13c) | 3-(1-[1,3]oxazolo[5,4- b]pyridin-2- ylpyrrolidin-3-yl)-3-[4- (7H-pyrrolo[2,3- d]pyrimidin-4-yl)-1H- pyrazol-1- yl]propanenitrile | 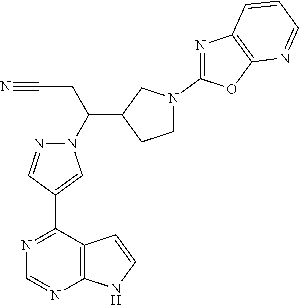 [SMILES: N#CCC(C1CCN(c2nc3cccnc3o2)C1)n1cc(-c2ncnc3[nH]ccc23)cn1] | + | >10 |
| 8 | US 2011/ 0059951 (Example 12) | 4-[(4-{3-cyano-2-[4- (7H-pyrrolo[2,3- d]pyrimidin-4-yl)-1H- pyrazol-1- yl]propyl}piperazin-1- yl)carbanoyl]-3- fluorobenzonitrile | 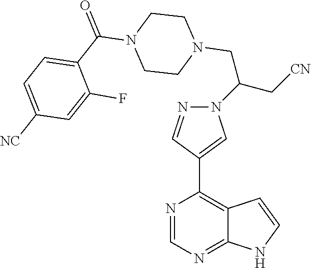 [SMILES: N#CCC(CN1CCN(C(=O)c2ccc(C#N)cc2F)CC1)n1cc(-c2ncnc3[nH]ccc23)cn1] | + | >10 |
| 9 | US 2011/ 0059951 (Example 13) | 4-[(4-{3-cyano-2-[3- (7H-pyrrolo[2,3- d]pyrimidin-4-yl)-1H- pyrrol-1- yl]propyl}piperazin-1- yl)carbonyl]-3- fluorobenzonitrile | 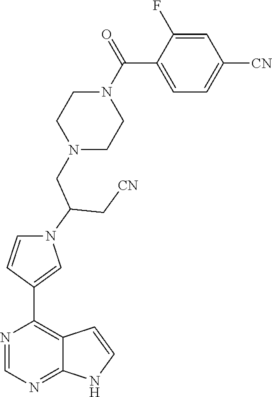 [SMILES: N#CCC(CN1CCN(C(=O)c2ccc(C#N)cc2F)CC1)n1ccc(-c2ncnc3[nH]ccc23)c1] | + | >10 |
| 10 | US 2012/ 0149681 (Example 7b) | [trans-1-[4-(7H- pyrrolo[2,3- d]pyrimidin-4-yl)-1H- pyrazol-1-yl]-3-(4-{[2- (trifluoromethyl)pyrimi- din-4- yl]carbonyl}piperazin- 1- yl)cyclobutyl]acetonitrile | 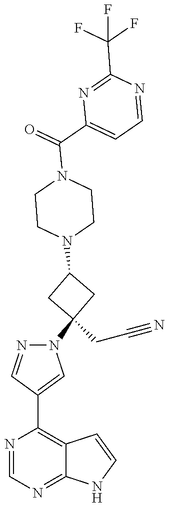 [SMILES: N#CC[C@]1(n2cc(-c3ncnc4[nH]ccc34)cn2)C[C@H](N2CCN(C(=O)c3ccnc(C(F)(F)F)n3)CC2)C1] | + | >10 |
| 11 | US 2012/ 0149681 (Example 157) | {trans-3-(4-{[4-[(3- hydroxyazetidin-1- yl)methyl]-6- (trifluoromethyl)pyridin- 2-yl]oxy}piperidin-1- yl)-1-[4-(7H- pyrrolo[2,3- d]pyrimidin-4-yl)-1H- pyrazol-1- yl]cyclobutyl}acetonitrile | 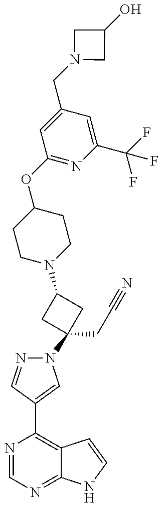 [SMILES: N#CC[C@]1(n2cc(-c3ncnc4[nH]ccc34)cn2)C[C@H](N2CCC(Oc3cc(CN4CC(O)C4)cc(C(F)(F)F)n3)CC2)C1] | + | >10 |
| 12 | US 2012/ 0149681 (Example 161) | {trans-3-(4-{[4-{[(2S)- 2- (hydroxymethyl)pyrroli- din-1-yl]methyl}-6- (trifluoromethyl)pyridin- 2-yl]oxy}piperidin-1- yl)-1-[4-(7H- pyrrolo[2,3- d]pyrimidin-4-yl)-1H- pyrazol-1- yl]cyclobutyl}acetonitrile | 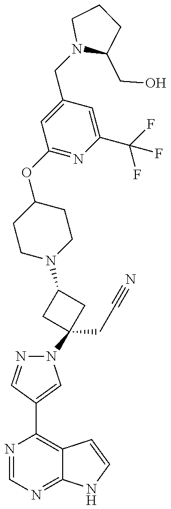 [SMILES: N#CC[C@]1(n2cc(-c3ncnc4[nH]ccc34)cn2)C[C@H](N2CCC(Oc3cc(CN4CCC[C@H]4CO)cc(C(F)(F)F)n3)CC2)C1] | + | >10 |
| 13 | US 2012/ 0149681 (Example 162) | {trans-3-(4-{[4-{[(2R)- 2- (hydroxymethyl)pyrroli- din-1-yl]methyl}-6- (trifluoromethyl)pyridin- 2-yl]oxy}piperidin-1- yl)-1-[4-(7H- pyrrolo[2,3- d]pyrimidin-4-yl)-1H- pyrazol-1- yl]cyclobutyl}acetonitrile | 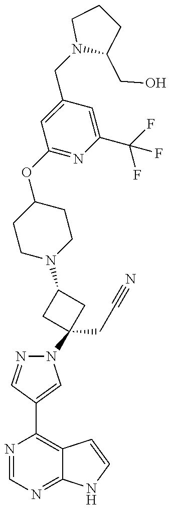 [SMILES: N#CC[C@]1(n2cc(-c3ncnc4[nH]ccc34)cn2)C[C@H](N2CCC(Oc3cc(CN4CCC[C@@H]4CO)cc(C(F)(F)F)n3)CC2)C1] | + | >10 |
| 14 | US 2012/ 0149682 (Example 20)^{b} | 4-(4-{3- [(dimethylamino)methyl]- 5- fluorophenoxy}piperidin- 1-yl)-3-[4-(7H- pyrrolo[2,3- d]pyrimidin-4-yl)-1H- pyrazol-1- yl]butanenitrile | 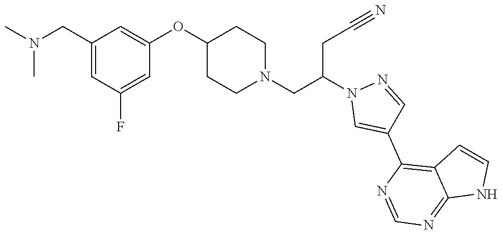 [SMILES: CN(C)Cc1cc(F)cc(OC2CCN(CC(CC#N)n3cc(-c4ncnc5[nH]ccc45)cn3)CC2)c1] | + | >10 |
| 15 | US 2013/ 0018034 (Example 18) | 5-{3-(cyanomethyl)-3- [4-(7H-pyrrolo[2,3- d]pyrimidin-4-yl)-1H- pyrazol-1-yl]azetidin-1- yl}-N- isopropylpyrazine-2- carboxamide | 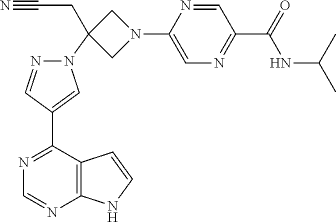 [SMILES: CC(C)NC(=O)c1cnc(N2CC(CC#N)(n3cc(-c4ncnc5[nH]ccc45)cn3)C2)cn1] | + | >10 |
| 16 | US 2013/ 0018034 (Example 28) | 4-{3-(cyanomethyl)-3- [4-(7H-pyrrolo[2,3- d]pyrimidin-4-yl)-1H- pyrazol-1-yl]azetidin-1- yl}-2,5-difluoro-N- [(1S)-2,2,2-trifluoro-1- methylethyl)benzamide | 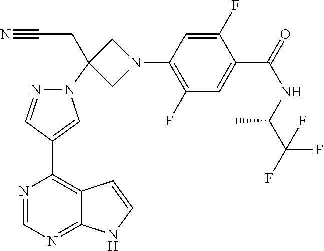 [SMILES: C[C@H](NC(=O)c1cc(F)c(N2CC(CC#N)(n3cc(-c4ncnc5[nH]ccc45)cn3)C2)cc1F)C(F)(F)F] | + | >10 |
| 17 | US 2013/ 0018034 (Example 34) | 5-{3-(cyanomethyl)-3- [4-(1H-pyrrolo[2,3- b]pyridin-4-yl)-1H- pyrazol-1-yl]azetidin-1- yl}-N- isopropylpyrazine-2- carboxamide | 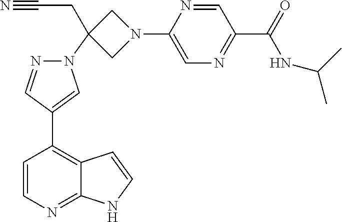 [SMILES: CC(C)NC(=O)c1cnc(N2CC(CC#N)(n3cc(-c4ccnc5[nH]ccc45)cn3)C2)cn1] | + | >10 |
| 18 | US 2013/ 0045963 (Example 45) | {1-(cis-4-{[6-(2- hydroxyethyl)-2- (trifluoromethyl)pyrimi- din-4- yl]oxy}cyclohexyl)-3- [4-(7H-pyrrolo[2,3- d]pyrimidin-4-yl)-1H- pyrazol-1-yl]azetidin-3- yl}acetonitrile | 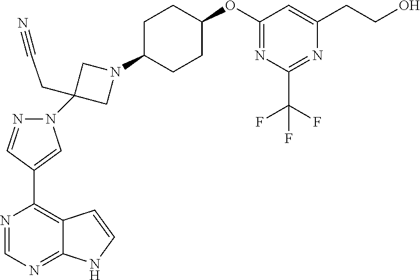 [SMILES: N#CCC1(n2cc(-c3ncnc4[nH]ccc34)cn2)CN([C@H]2CC[C@@H](Oc3cc(CCO)nc(C(F)(F)F)n3)CC2)C1] | + | >10 |
| 19 | US 2013/ 0045963 (Example 65) | {1-(cis-4-{[4- [(ethylamino)methyl]-6- (trifluoromethyl)pyridin- 2-yl]oxy}cyclohexyl)- 3-[4-(7H-pyrrolo[2,3- d]pyrimidin-4-yl)-1H- pyrazol-1-yl]azetidin-3- yl}acetonitrile | 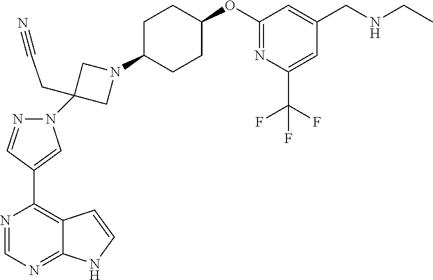 [SMILES: CCNCc1cc(O[C@H]2CC[C@@H](N3CC(CC#N)(n4cc(-c5ncnc6[nH]ccc56)cn4)C3)CC2)nc(C(F)(F)F)c1] | + | >10 |
| 20 | US 2013/ 0045963 (Example 69) | {1-(cis-4-{[4-(1- hydroxy-1- methylethyl)-6- (trifluoromethyl)pyridin- 2-yl]oxy}cyclohexyl)- 3-[4-(7H-pyrrolo[2,3- d]pyrimidin-4-yl)-1H- pyrazol-1-yl]azetidin-3- yl}acetonitrile | 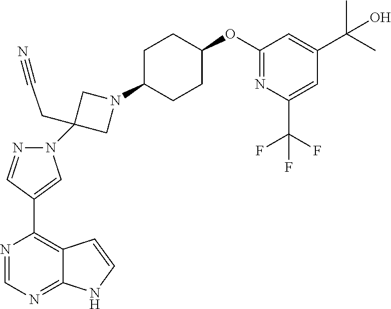 [SMILES: CC(C)(O)c1cc(O[C@H]2CC[C@@H](N3CC(CC#N)(n4cc(-c5ncnc6[nH]ccc56)cn4)C3)CC2)nc(C(F)(F)F)c1] | + | >10 |
| 21 | US 2013/ 0045963 (Example 95) | {1-(cis-4-{[4-{[(3R)-3- hydroxypyrrolidin-1- yl]methyl}-6- (trifluoromethyl)pyridin- 2-yl]oxy}cyclohexyl)- 3-[4-(7H-pyrrolo[2,3- d]pyrimidin-4-yl)-1H- pyrazol-1-yl]azetidin-3- yl}acetonitrile | 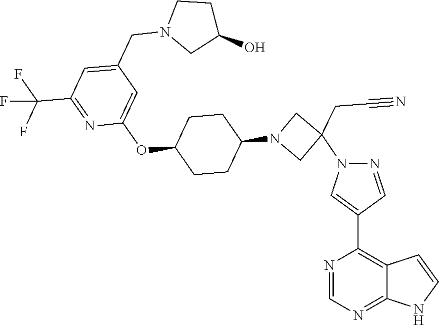 [SMILES: N#CCC1(n2cc(-c3ncnc4[nH]ccc34)cn2)CN([C@H]2CC[C@@H](Oc3cc(CN4CC[C@@H](O)C4)cc(C(F)(F)F)n3)CC2)C1] | + | >10 |
| 22 | US 2013/ 0045963 (Example 95) | {1-(cis-4-{[4-{[(3S)-3- hydroxypyrrolidin-1- yl]methyl}-6- (trifluoromethyl)pyridin- 2-yl]oxy}cyclohexyl)- 3-[4-(7H-pyrrolo[2,3- d]pyrimidin-4-yl)-1H- pyrazol-1-yl]azetidin-3- yl}acetonitrile | 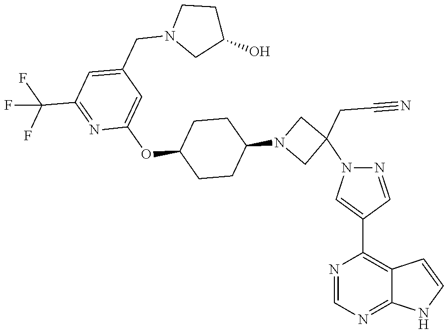 [SMILES: N#CCC1(n2cc(-c3ncnc4[nH]ccc34)cn2)CN([C@H]2CC[C@@H](Oc3cc(CN4CC[C@H](O)C4)cc(C(F)(F)F)n3)CC2)C1] | + | >10 |
| 23 | US 2014/ 0005166 (Example 1) | {trans-3-(4-{[4-({[(1S)- 2-hydroxy-1- methylethyl]amino}meth- yl)-6- (trifluoromethyl)pyridin- 2-yl]oxy}piperidin-1- yl)-1-[4-(7H- pyrrolo[2,3- d]pyrimidin-4-yl)-1H- pyrazol-1- yl]cyclobutyl}acetonitrile | 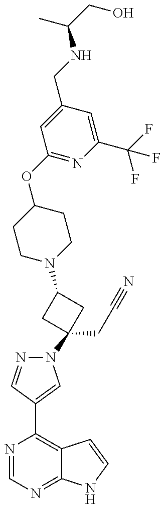 [SMILES: C[C@@H](CO)NCc1cc(OC2CCN([C@H]3C[C@](CC#N)(n4cc(-c5ncnc6[nH]ccc56)cn4)C3)CC2)nc(C(F)(F)F)c1] | + | >10 |
| 24 | US 2014/ 0005166 (Example 14) | {trans-3-(4-{[4-({[(2R)- 2- hydroxypropyl]amino} methyl)-6- (trifluoromethyl)pyridin- 2-yl]oxy}piperidin-1- yl)-1-[4-(7H- pyrrolo[2,3- d]pyrimidin-4-yl)-1H- pyrazol-1- yl]cyclobutyl}acetonitrile | 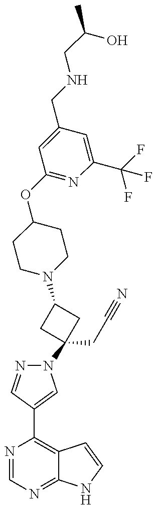 [SMILES: C[C@@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 25 | US 2014/ 0005166 (Example 15) | {trans-3-(4-{[4-({[(2S)- 2- hydroxypropyl]amino} methyl)-6- (trifluoromethyl)pyridin- 2-yl]oxy}piperidin-1- yl)-1-[4-(7H- pyrrolo[2,3- d]pyrimidin-4-yl)-1H- pyrazol-1- yl]cyclobutyl}acetonitrile | 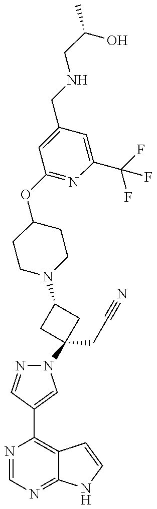 [SMILES: C[C@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 26 | US 2014/ 0005166 (Example 20) | {trans-3-(4-{[4-(2- hydroxyethyl)-6- (trifluoromethyl)pyridin- 2-yl]oxy}piperidin-1- yl)-1-[4-(7H- pyrrolo[2,3- d]pyrimidin-4-yl)-1H- pyrazol-1- yl]cyclobutyl}acetonitrile | 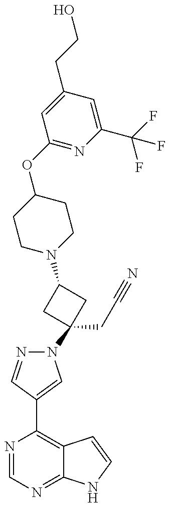 [SMILES: N#CC[C@]1(n2cc(-c3ncnc4[nH]ccc34)cn2)C[C@H](N2CCC(Oc3cc(CCO)cc(C(F)(F)F)n3)CC2)C1] | + | >10 |

+ means <10 nM (see Example A for assay conditions)
++ means ≤100 nM (see Example A for assay conditions)
+++ means ≤300 nM (see Example A for assay conditions)
^{a}Data for enantiomer 1
^{b}Data for enantiomer 2

In some embodiments, the JAK1 inhibitor is {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl} acetonitrile, or a pharmaceutically acceptable salt thereof.

In some embodiments, the JAK1 inhibitor is {1-{1[3-fluoro-2-(trifluoromethyl)isonicotinoyl]piperidin-4-yl}-3 -[4-(7H-pyrrolo[2,3-d]pyrimidin-4-yl)-1H-pyrazol-1-yl]azetidin-3-yl] acetonitrileadipicacid salt.

The synthesis and preparation of {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl} acetonitrile and the adipic acid salt of the same can be found, e.g., in US Patent Publ. No. 2011/0224190, filed Mar. 9, 2011, US Patent Publ. No. 2013/0060026, filed Sep. 6, 2012, and US Patent Publ. No. 2014/0256941, filed Mar. 5, 2014, each of which is incorporated herein by reference in its entirety.

In some embodiments, the JAK1 inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof.

In some embodiments, the JAK1 inhibitor is 4-[3-(cyanomethyl)-3-(3′,5′-dimethyl-1H,1′H-4,4′-bipyrazol-1-yl)azetidin-1- yl]-2,5-difluoro-N-[(1S)-2,2,2-trifluoro-1-methylethyl]benzamide phosphoric acid salt.

In some embodiment, the JAK1 is 4-[3-(cyanomethyl)-3-(3′,5′-dimethyl-1H, 2′H-4,4′-bipyrazol-1-yl)azetidin-1-yl]-2,5-difluoro-N-[(1S)-2,2,2-trifluoro-1-methylethyl]benzamide hydrochloric acid salt.

In some embodiment, the JAK1 is 4-[3-(cyanomethyl)-3-(3′,5′-dimethyl-1H, 1′H-4,4′-bipyrazol-1-yl)azetidin-1-yl]-2,5-difluoro-N-[(1S)-2,2,2-trifluoro-1-methylethyl]benzamide hydrobromic acid salt.

In some embodiment, the JAK1 is 4-[3-(cyanomethyl)-3-(3′,5′-dimethyl-1H, 1′H-4,4′-bipyrazol-1-yl)azetidin-1-yl]-2,5-difluoro-N-[(1S)-2,2,2-trifluoro-1-methylethyl]benzamide sulfuric acid salt.

The synthesis and preparation of 4-[3-(cyanomethyl)-3-(3′,5′-dimethyl-1H,1′H-4,4′-bipyrazol-1-yl)azetidin-1-yl]-2,5- difluoro-N-[(1S)-2,2,2-trifluoro-1-methylethyl]benzamide and the phosphoric acid salt of the same can be found, e.g., in US Patent Publ. No. US 2014/0343030, filed May 16, 2014, which is incorporated herein by reference in its entirety.

In some embodiments, the JAK1 inhibitor is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile, or a pharmaceutically acceptable salt thereof.

In some embodiments, the JAK1 inhibitor is ((2R,5S)-5-{2-[(1R)-1-hydroxyethyl]-1H-imidazo[4,5-d]thieno[3,2-b]pyridin-1-yl]tetrahydro-2H-pyran-2-yl)acetonitrile monohydrate.

Synthesis of ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile and characterization of the anhydrous and monohydrate forms of the same are described in US Patent Publ. No. 2014/0121198, filed Oct. 31, 2013 and US Patent Publ. No. 2015/0344497, filed Apr. 29, 2015, each of which is incorporated herein by reference in its entirety.

In some embodiments, the compounds of Table 1 are prepared by the synthetic procedures described in US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, JAK1 inhibitor is selected from the compounds, or pharmaceutically acceptable salts thereof, of US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, the JAK1 inhibitor is a compound of Formula I

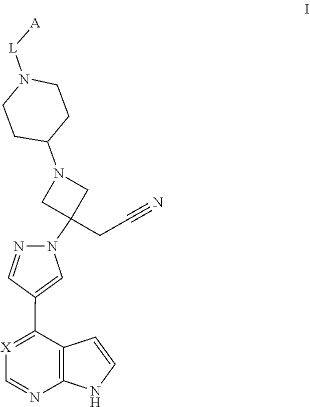

or a pharmaceutically acceptable salt thereof, wherein:

X is N or CH;

L is C(═O) or C(λO)NH;

A is phenyl, pyridinyl, or pyrimidinyl each of which is optionally substituted with 1 or 2 independently selected le groups; and

each R¹ is, independently, fluoro, or trifluoromethyl.

In some embodiments, the compound of Formula I is { 1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl} acetonitrile, or a pharmaceutically acceptable salt thereof.

In some embodiments, the compound of Formula I is 4-{3-(Cyanomethyl)-3-[4(7H-pyrrolo[2,3 -d]pyrimidin-4-yl)-1H-pyrazol-1-yl]azetidin-1-yl }-N-[4-fluoro-2-trifluoromethyl)phenyl]piperidine-1-carboxamide, or a pharmaceutically acceptable salt thereof.

In some embodiments, the compound of Formula I is [3-[4-(7H-pyrrolo[2,3-d]pyrimidin-4-yl)-1H-pyrazol-1-yl]-1-(1-{[2-(trifluoromethyl)pyrimidin-4-yl]carbonylIpiperidin-4-yl)azetidin-3-yl]acetonitrile, or a pharmaceutically acceptable salt thereof.

In some embodiments, the JAK1 inhibitor is a compound of Formula II

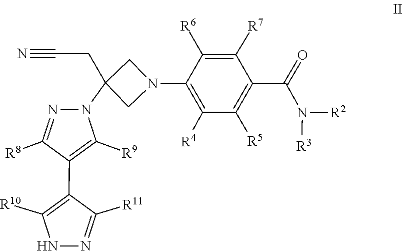

or a pharmaceutically acceptable salt thereof, wherein:

R² is C₁₋₆ alkyl, C₁₋₆ haloalkyl, C₃₋₆ cycloalkyl, or C₃₋₆ cycloalkyl-C₁₋₃ alkyl, wherein said C₁₋₆ alkyl, C₃₋₆ cycloalkyl, and C₃₋₆ cycloalkyl-C₁₋₃ alkyl, are each optionally substituted with 1, 2, or 3 substituents independently selected from fluoro, —CF₃, and methyl;

R³ is H or methyl;

R⁴ is H, F, or Cl;

R⁵ is H or F;

R⁶ is H or F;

R⁷ is H or F;

R⁸ is H or methyl;

R⁹ is H or methyl;

R¹⁰ is H or methyl; and

R¹¹ is H or methyl.

In some embodiments, the compound of Formula II is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide , or a pharmaceutically acceptable salt thereof.

In some embodiments, the JAK1 inhibitor is a compound of Formula III

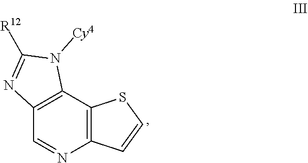

or a pharmaceutically acceptable salt thereof, wherein:

Cy⁴ is a tetrahydro-2H-pyran ring, which is optionally substituted with 1 or 2 groups independently selected from CN, OH, F, Cl, C₁₋₃alkyl, C₁₋₃ haloalkyl, CN-C₁₋₃ alkyl, HO—C₁₋₃ alkyl, amino, C₁₋₃ alkylamino, and di(C₁₋₃ alkyl)amino, wherein said C₁₋₃ alkyl and di(C₁₋₃alkyl)amino is optionally substituted with 1, 2, or 3 substituents independently selected from F, Cl, C₁₋₃ alkylaminosulfonyl, and C₁₋₃ alkylsulfonyl; and

R¹² is —CH₂—OH, —CH(CH₃)—OH, or —CH₂—NHSO₂CH₃.

In some embodiments, the compound of Formula III is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile, or a pharmaceutically acceptable salt thereof.

In some embodiments, the inhibitor of JAK1 can be an isotopically-labeled compound, or a pharmaceutically acceptable salt thereof. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³ H (also written as T for tritium), ¹¹C, ¹³C, ¹⁴C, ¹³N, ¹⁵N, ¹⁵O, ¹⁷O, ¹⁸O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms, such as —CD₃ being substituted for —CH₃).

One or more constituent atoms of the compounds described herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

Accordingly, in some embodiments, the inhibitor of JAK1 is a compound, wherein one or more hydrogen atoms in the compound are replaced by deuterium atoms, or a pharmaceutically acceptable salt thereof.

As used herein, the phrase “optionally substituted” means unsubstituted or substituted. As used herein, the term “substituted” means that a hydrogen atom is removed and replaced by a substituent. It is to be understood that substitution at a given atom is limited by valency.

As used herein, the term “Cn-m alkyl”, employed alone or in combination with other terms, refers to a saturated hydrocarbon group that may be straight-chain or branched, having n to m carbon atoms. In some embodiments, the alkyl group contains 1 to 6, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, and the like.

As used herein, the term “alkylene”, employed alone or in combination with other terms, refers to a divalent alkyl linking group, which can be branched or straight-chain, where the two substituents may be attached any position of the alkylene linking group. Examples of alkylene groups include, but are not limited to, ethan-1,2-diyl, propan-1,3-diyl, propan-1,2-diyl, and the like.

As used herein, the term “HO—C₁₋₃-alkyl” refers to a group of formula -alkylene-OH, wherein said alkylene group has 1 to 3 carbon atoms.

As used herein, the term “CN—C₁₋₃ alkyl” refers to a C₁₋₃ alkyl substituted by a cyano group.

As used herein, the term “amino” refers to a group of formula —NH₂.

As used herein, the term “di(C₁₋₃-alkyl)amino” refers to a group of formula —N(alkyl)₂, wherein the two alkyl groups each has, independently, 1 to 3 carbon atoms.

As used herein, the term “C₁₋₃ alkylamino” refers to a group of formula —NH(alkyl), wherein the alkyl group has 1 to 3 carbon atoms.

As used herein, the term “di(C₁₋₃ alkyl)aminosulfonyl” refers to a group of formula —SO₂N(alkyl)₂, wherein each alkyl group independently has 1 to 3 carbon atoms.

As used herein, the term “C₁₋₃ alkylsulfonyl” refers to a group of formula —S(O)₂— alkyl, wherein the alkyl group has 1 to 3 carbon atoms.

As used herein, “halo” or “halogen”, employed alone or in combination with other terms, includes fluoro, chloro, bromo, and iodo. In some embodiments, the halo group is fluoro or chloro.

As used herein, the term “Cₙ₋ₘ haloalkyl”, employed alone or in combination with other terms, refers to a Cₙ₋ₘ alkyl group having up to {2(n to m)+1} halogen atoms which may either be the same or different. In some embodiments, the halogen atoms are fluoro atoms. In some embodiments, the alkyl group has 1-6 or 1-3 carbon atoms. Example haloalkyl groups include CF₃, C₂F₅, CHF₂, CCl₃, CHCl₂, C₂Cl₅, and the like. In some embodiments, the haloalkyl group is a fluoroalkyl group.

As used herein, the term “C₁₋₃ fluoroalkyl” refers to a C₁₋₃ alkyl group that may be partially or completely substituted by fluoro atoms.

As used herein, the term “C₃₋₆ cycloalkyl”, employed alone or in combination with other terms, refers to a non-aromatic monocyclic hydrocarbon moiety, having 3-6 carbon atoms, which may optionally contain one or more alkenylene groups as part of the ring structure. One or more ring-forming carbon atoms of a cycloalkyl group can be oxidized to form carbonyl linkages. Exemplary C₃₋₆ cycloalkyl groups include cyclopropyl, cyclobutyl, cyclopentyl, cyclohexyl, cyclopentenyl, cyclohexenyl, cyclohexadienyl, and the like. In some embodiments, the cycloalkyl group is cyclopropyl, cyclobutyl, cyclopentyl, or cyclohexyl.

As used herein, the term “C₃₋₆ cycloalkyl-C₁₋₃ alkyl” refers to a group of formula —C₁₋₃ alkylene-C₃₋₆ cycloalkyl.

The compounds described herein can be asymmetric (e.g., having one or more stereocenters). All stereoisomers, such as enantiomers and diastereomers, are intended unless otherwise indicated. Compounds that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present application. Cis and trans geometric isomers of the compounds of the present application are described and may be isolated as a mixture of isomers or as separated isomeric forms. In some embodiments, the compound has the (R)-configuration. In some embodiments, the compound has the (S)-configuration.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as (β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds described herein include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide-imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution. For example, it will be recognized that the following pyrazole ring may form two tautomers:

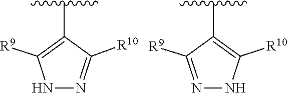

It is intended that the claims cover both tautomers.

All compounds, and pharmaceutically acceptable salts thereof, can be found together with other substances such as water and solvents (e.g. hydrates and solvates) or can be isolated.

In some embodiments, the compounds described herein, or salts thereof, are substantially isolated. By “substantially isolated” is meant that the compound is at least partially or substantially separated from the environment in which it was formed or detected. Partial separation can include, for example, a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds described herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “contacting” refers to the bringing together of indicated moieties in an in vitro system or an in vivo system. For example, “contacting” a JAK with a compound of the invention includes the administration of a compound of the present application to an individual or patient, such as a human, having a JAK, as well as, for example, introducing a compound of the invention into a sample containing a cellular or purified preparation containing the JAK.

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans. In some embodiments, the “subject,” “individual,” or “patient” is in need of said treatment.

In some embodiments, the inhibitors are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease; or (3) preventing the disease, condition or disorder in an individual who may be predisposed to the disease, condition or disorder but does not yet experience or display the pathology or symptomatology of the disease. In some embodiments, treating refers to inhibiting or ameliorating the disease. In some embodiments, treating is preventing the disease.

### Combination Therapies

The methods described herein can further comprise administering one or more additional therapeutic agents. The one or more additional therapeutic agents can be administered to a patient simultaneously or sequentially.

In some embodiments, the additional therapeutic agent includes an antibiotic, antiviral, antifungal, anesthetic, anti-inflammatory agents including steroidal and non-steroidal anti-inflammatories, and anti-allergic agents. Examples of suitable medicaments include aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; naphthyridine; sulfonamides; polymyxin; chloramphenicol; neomycin; paramomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; diclofenac; flurbiprofen; ketorolac; suprofen; cromolyn; lodoxamide; levocabastin; naphazoline; antazoline; pheniramine; or azalide antibiotic.

In some embodiments, the additional therapeutic agent includes an antihistamine. The antihistamine is a second-generation HI antihistamine.

In some embodiments, the additional therapeutic agent is an antibiotic. In some embodiments, the antibiotic is clindamycin, doxycycline, minocycline, trimethoprim-sulfamethoxazole, erythromycin, metronidazole, rifampin, moxifloxacin, dapsone, or a combination thereof. In some embodiments, the antibiotic is clindamycin, doxycycline, minocycline, trimethoprim-sulfamethoxazole, or erythromycin in combination with metronidazole. In some embodiments, the antibiotic is a combination of rifampin, moxifloxacin, and metronidazole. In some embodiments, the antibiotic is a combination of moxifloxacin and rifampin.

In some embodiments, the additional therapeutic agent is a retinoid. In some embodiments, the retinoid is etretinate, acitretin, or isotretinoin.

In some embodiments, the additional therapeutic agent is a steroid. In some embodiments, the additional therapeutic agent is a corticosteroid. In some embodiments, the steroid is such as triamcinolone, dexamethasone, fluocinolone, cortisone, prednisone, prednisolone, or flumetholone.

In some embodiments, the additional therapeutic agent is an anti-TNF-alpha agent. In some embodiments, the anti-TNF-alpha agent is an anti-TNF-alpha antibody. In some embodiments, the anti-TNF-alpha agent is infliximab or etanercept, or adalimumab.

In some embodiments, the additional therapeutic agent is an immunosuppressant. In some embodiments, the immunosuppressant is methotrexate or cyclosporin A. In some embodiments, the immunosuppressant is mycophenolate mofetil or mycophenolate sodium.

In some embodiments, the additional therapeutic agent is finasteride, metformin, adapalene or azelaic acid.

In some embodiments, the method further comprises administering an additional therapeutic agent selected from IMiDs, an anti-IL-6 agent, a hypomethylating agent, and a biologic response modifier (BRM).

Generally, a BRM is a substances made from living organisms to treat disease, which may occur naturally in the body or may be made in the laboratory. Examples of BRMs include IL-2, interferon, various types of colony-stimulating factors (CSF, GM-CSF, G-CSF), monoclonal antibodies such as abciximab, etanercept, infliximab, omalizumab, rituximab, trasturzumab, and high dose ascorbate.

In some embodiments, the hypomethylating agent is a DNA methyltransferase inhibitor. In some embodiments, the DNA methyltransferase inhibitor is selected from 5 azacytidine and decitabine.

Generally, IMiDs are as immunomodulatory agents. In some embodiments, the IMiD is selected from thalidomide, lenalidomide, pomalidomide, CC-11006, and CC-10015.

In some embodiments, the method further comprises administering an additional therapeutic agent selected from anti-thymocyte globulin, recombinant human granulocyte colony-stimulating factor (G CSF), granulocyte-monocyte CSF (GM-CSF), an erythropoiesis-stimulating agent (ESA), and cyclosporine.

In some embodiments, the method further comprises administering an additional JAK inhibitor to the patient. In some embodiments, the additional JAK inhibitor is ruxolitinib, barcitinib, tofacitinib, oclacitinib, filgotinib, gandotinib, lestaurtinib, momelotinib, PF-04965842, upadacitinib, peficitinib, fedratinib, cucurbitacin I, or CHZ868. Additional JAK inhibitors may include ATI-50002 (JAK1/3 selective). Additional JAK inhibitors may include PF-06651600 (JAK3 selective). Additional JAK inhibitors may include PF06700841 (JAK1/TYK2 selective). Additional JAK inhibitors may include TYK2 selective inhibitors.

In some embodiments, the additional therapeutic agent is selected from antioxidants. Antioxidants may be selected from pseudocatalase, vitamin E, vitamin C, ubiquinone, lipoic acid, Polypodium leucotomos, catalase/superoxide dismutase combination, and Ginkgo biloba. In some embodiments, antioxidants may be further administered in combination with phototherapy. The administration of antioxidants during or before phototherapy aims to counteract the oxidative stress induced by UV radiation itself, increasing the phototherapy effectiveness.

In some embodiments, the additional therapeutic agent includes anti-histamines.

In some embodiments, the additional therapeutic agent is selected from topical corticosteroids, immunomodulators, calcineurin inhibitors, and phototherapy. In some embodiments, the additional therapies are systemic steroids.

One or more additional pharmaceutical agents such as, for example, anti-inflammatory agents, immunosuppressants, as well as PI3Kδ, mTor, Bcr-Abl, Flt-3, RAF and FAK kinase inhibitors such as, for example, those described in WO 2006/056399, which is incorporated herein by reference in its entirety, or other agents can be used in combination with the compounds described herein for treatment of JAK-associated diseases, disorders or conditions. The one or more additional pharmaceutical agents can be administered to a patient simultaneously or sequentially.

Example Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491, all of which are incorporated herein by reference in their entirety.

Example suitable Flt-3 inhibitors include compounds, and their pharmaceutically acceptable salts, as disclosed in WO 03/037347, WO 03/099771, and WO 04/046120, all of which are incorporated herein by reference in their entirety.

Example suitable RAF inhibitors include compounds, and their pharmaceutically acceptable salts, as disclosed in WO 00/09495 and WO 05/028444, both of which are incorporated herein by reference in their entirety.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402, all of which are incorporated herein by reference in their entirety.

In some embodiments, one or more of the compounds of the invention can be used in combination with one or more other kinase inhibitors including imatinib, particularly for treating patients resistant to imatinib or other kinase inhibitors.

In some embodiments, the additional therapeutic agent is fluocinolone acetonide (Retisert®), or rimexolone (AL-2178, Vexol, Alcon).

In some embodiments, the additional therapeutic agent is cyclosporine (Restasis®).

In some embodiments, the additional therapeutic agent is selected from Dehydrex™ (Holles Labs), Civamide (Opko), sodium hyaluronate (Vismed, Lantibio/TRB Chemedia), cyclosporine (ST-603, Sirion Therapeutics), ARG101(T) (testosterone, Argentis), AGR1012(P) (Argentis), ecabet sodium (Senju-Ista), gefarnate (Santen), 15-(s)-hydroxyeicosatetraenoic acid (15(S)-RETE), cevilemine, doxycycline (ALTY-0501, Alacrity), minocycline, iDestrinT™ (NP50301, Nascent Pharmaceuticals), cyclosporine A (Nova22007, Novagali), oxytetracycline (Duramycin, MOLI1901, Lantibio), CF101 (2S,3S,4R,5R)-3,4-dihydroxy-546-[(3-iodophenyl)methylamino]purin-9-yl]-N-methyl-oxolane-2-carbamyl, Can-Fite Biopharma), voclosporin (LX212 or LX214, Lux Biosciences), ARG103 (Agentis), RX-10045 (synthetic resolvin analog, Resolvyx), DYN15 (Dyanmis Therapeutics), rivoglitazone (DE011, Daiichi Sanko), TB4 (RegeneRx), OPH-01 (Ophtalmis Monaco), PCS101 (Pericor Science), REV1-31 (Evolutec), Lacritin (Senju), rebamipide (Otsuka-Novartis), OT-551 (Othera), PAI-2 (University of Pennsylvania and Temple University), pilocarpine, tacrolimus, pimecrolimus (AMS981, Novartis), loteprednol etabonate, rituximab, diquafosol tetrasodium (INS365, Inspire), KLS-0611 (Kissei Pharmaceuticals), dehydroepiandrosterone, anakinra, efalizumab, mycophenolate sodium, etanercept (Embrel®), hydroxychloroquine, NGX267 (TorreyPines Therapeutics), actemra, gemcitabine, oxaliplatin, L-asparaginase, or thalidomide.

In some embodiments, the additional therapeutic agent is an anti-angiogenic agent, cholinergic agonist, TRP-1 receptor modulator, a calcium channel blocker, a mucin secretagogue, MUC1 stimulant, a calcineurin inhibitor, a corticosteroid, a P2Y2 receptor agonist, a muscarinic receptor agonist, an mTOR inhibitor, another JAK inhibitor, Bcr-Abl kinase inhibitor, Flt-3 kinase inhibitor, RAF kinase inhibitor, and FAK kinase inhibitor such as, for example, those described in WO 2006/056399, which is incorporated herein by reference in its entirety. In some embodiments, the additional therapeutic agent is a tetracycline derivative (e.g., minocycline or doxycline). In some embodiments, the additional therapeutic agent binds to FKBP12.

In some embodiments, the additional therapeutic agent is an alkylating agent or DNA cross-linking agent; an anti-metabolite/demethylating agent (e.g., 5-flurouracil, capecitabine or azacitidine); an anti-hormone therapy (e.g., hormone receptor antagonists, SERMs, or aromotase inhibitor); a mitotic inhibitor (e.g. vincristine or paclitaxel); an topoisomerase (I or II) inhibitor (e.g. mitoxantrone and irinotecan); an apoptotic inducers (e.g. ABT-737); a nucleic acid therapy (e.g. antisense or RNAi); nuclear receptor ligands (e.g., agonists and/or antagonists: all-trans retinoic acid or bexarotene); epigenetic targeting agents such as histone deacetylase inhibitors (e.g. vorinostat), hypomethylating agents (e.g. decitabine); regulators of protein stability such as Hsp90 inhibitors, ubiquitin and/or ubiquitin like conjugating or deconjugating molecules; or an EGFR inhibitor (erlotinib).

### Pharmaceutical Formulations and Dosage Forms

When employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

In some embodiments, the administration is topical. In some embodiments, the administration is topical administration to the skin.

In some embodiments, the administration is oral.

This invention also includes pharmaceutical compositions which contain, as the active ingredient, the compound of the invention or a pharmaceutically acceptable salt thereof, in combination with one or more pharmaceutically acceptable carriers (excipients). In some embodiments, the composition is suitable for topical administration. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier. In some embodiments, the composition comprises at least one compound described herein, or a pharmaceutically acceptable salt thereof, and at least one component selected from microcrystalline cellulose, lactose monohydrate, hydroxypropyl methylcellulose, and polyethylene oxide. In some embodiments, the composition comprises at least one compound described herein, or a pharmaceutically acceptable salt thereof, and microcrystalline cellulose, lactose monohydrate, and hydroxypropyl methylcellulose. In some embodiments, the composition comprises at least one compound described herein, or a pharmaceutically acceptable salt thereof, and microcrystalline cellulose, lactose monohydrate, and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 1 to about 1,000 mg, from about 1 mg to about 100 mg, from 1 mg to about 50 mg, and from about 1 mg to 10 mg of active ingredient. Preferably, the dosage is from about 1 mg to about 50 mg or about 1 mg to about 10 mg of active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions comprise from about 1 to about 1,000 mg, from about 1 mg to about 100 mg, from 1 mg to about 50 mg, and from about 1 mg to 10 mg of active ingredient. Preferably, the compositions comprise from about 1 mg to about 50 mg or about 1 mg to about 10 mg of active ingredient. One having ordinary skill in the art will appreciate that this embodies compounds or compositions containing about 1 mg to about 10 mg, about 1 mg to about 20 mg, about 1 mg to about 25 mg, about 1 mg to about 50 mg of the active ingredient.

In some embodiments, the dosage of the compound, or a pharmaceutically acceptable salt thereof, is about 10-90 mg on a free base basis. In some embodiments, the dosage of the compound, or a pharmaceutically acceptable salt thereof, is about 15-75 mg on a free base basis. In some embodiments, the dosage of the compound, or a pharmaceutically acceptable salt thereof, is 15, 30, 45 or 75 mg on a free base basis. In some embodiments, the dosage is 15, 30, 45 or 75 mg on a free base basis, of Compound 4 of Table 1, or a pharmaceutically acceptable salt thereof. In some embodiments, the dosage of the compound, or a pharmaceutically acceptable salt thereof, is 15 mg on a free base basis. In some embodiments, the dosage of the compound, or a pharmaceutically acceptable salt thereof, is 30 mg on a free base basis. In some embodiments, the dosage of the compound, or a pharmaceutically acceptable salt thereof, is 45 mg on a free base basis. In some embodiments, the dosage of the compound, or a pharmaceutically acceptable salt thereof, is 75 mg on a free base basis.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

Active compounds described herein such as JAK1 inhibitors may have a long half-life. The long half-life of JAK inhibitors provides good peak trough levels of availability once a steady state is reached (e.g., over 24 hour dosing). As opposed to JAK inhibitors, Omalizumab, for example, is often dosed at higher than studied levels for more refractory patients (600 mg per month).

For preparing solid compositions such as tablets, the principal active ingredient is mixed with a pharmaceutical excipient to form a solid preformulation composition containing a homogeneous mixture of a compound of the present application. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present application.

The tablets or pills of the present application can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present application can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the invention. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present application can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents, examples of which are listed hereinabove.

### Kits

The present application also includes pharmaceutical kits useful, for example, in the treatment and/or prevention of cytokine-related diseases or disorders, such as CRS, which include one or more containers containing a pharmaceutical composition comprising a therapeutically effective amount of a compound described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example A: In Vitro JAK Kinase Assay

JAK1 inhibitors that can be used for the treatment of cytokine-related diseases or disorders are tested for inhibitory activity of JAK targets according to the following in vitro assay described in Park et al., Analytical Biochemistry 1999, 269, 94-104. The catalytic domains of human JAK1 (a.a. 837-1142), JAK2 (a.a. 828-1132) and JAK3 (a.a. 781-1124) with an N-terminal His tag are expressed using baculovirus in insect cells and purified. The catalytic activity of JAK1, JAK2 or JAK3 was assayed by measuring the phosphorylation of a biotinylated peptide. The phosphorylated peptide was detected by homogenous time resolved fluorescence (HTRF). IC5os of compounds are measured for each kinase in the 40 microL reactions that contain the enzyme, ATP and 500 nM peptide in 50 mM Tris (pH 7.8) buffer with 100 mM NaCl, 5 mM DTT, and 0.1 mg/mL (0.01%) BSA. For the 1 mM IC50 measurements, ATP concentration in the reactions is 1 mM. Reactions are carried out at room temperature for 1 hour and then stopped with 20 μL 45 mM EDTA, 300 nM SA-APC, 6 nM Eu-Py20 in assay buffer (Perkin Elmer, Boston, MA). Binding to the Europium labeled antibody takes place for 40 minutes and HTRF signal was measured on a Fusion plate reader (Perkin Elmer, Boston, MA). The compounds in Table 1 were tested in this assay and shown to have the IC₅₀ values in Table 1

### Example B:

### Upregulated JAK-STAT Pathway Expression

RNA are isolated from formalin-fixed paraffin embedded (FFPE) skin biopsies from active disease, untreated, urticaria patients. RNA are processed using the nCounter autoimmune profiling codeset (770 genes) or the neuropathology profiling codeset (770 genes) (Nanostring, USA), according to the manufacturer's protocol. After an 18 h hybridization, the samples are run on an nCounter SPRINT Profiler (Nanostring, USA).

Data are analyzed using nSolver 4.0 Advanced Analysis software (Nanostring, USA). P-values are adjusted using the Benjamini-Yekutieli false discovery rate method.

### JAK1 Mediated Pharmacological Inhibition of PN Pathophysiology

Full-thickness cutaneous skin biopsies are obtained from untreated active disease, untreated, urticaria patients. A single 4 mm-punch biopsy is obtained from the same lesion for each patient and longitudinally divided into two pieces. Biopsies are cultured for 8 days in KBM media+CaCl2 and refreshed every 2-3 days. Compound or DMSO (control) is added to the cell culture media. Conditioned supernatants collected during prior to media refresh are stored at −80° C. for until subsequent analysis. On day 8, cultures are terminated and tissue RNA isolated for subsequent analysis. RNA are processed using the nCounter autoimmune profiling codeset (770 genes) or the neuropathology profiling codeset (770 genes) (Nanostring, USA), according to the manufacturer's protocol. After an 18 h hybridization, the samples are run on an nCounter SPRINT Profiler (Nanostring, USA). Data are analyzed using nSolver 4.0 Advanced Analysis software (Nanostring, USA). P-values are adjusted using the Benjamini-Yekutieli false discovery rate method.

### Example C

### Upregulated JAK-STAT Pathway Expression

Signature pathway analysis was performed on publicly available data from two published studies to help define the gene expression profile in CSU: Gimenez-Arnau et al. (https://pubmed.ncbi.nlm.nih.gov/28407332/) and Patel et al. (https://www.ncbi.nlm.nih.gov/pmc/articles/PMC4541630/) both of which are incorporated by reference. Analyzing these data against hallmark pathways, 22 predefined pathways were identified as upregulated in CSU patient skin, including the hallmark inflammatory response pathway, the hallmark interferon gamma response pathway, and the hallmark 1L6 JAK STAT3 signaling pathway.

### JAKJ Mediated Pharmacological Inhibition of CSU Pathophysiology

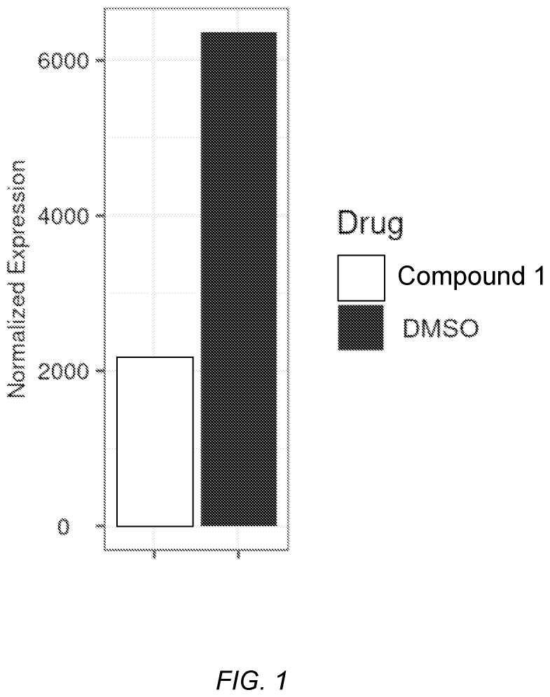

Full-thickness cutaneous skin biopsies were obtained from active disease, untreated, urticaria patients. A single 4mm-punch biopsy was obtained from the same lesion for each patient and longitudinally divided into two pieces. Biopsies were cultured for 24 hours in KBM media+CaCl₂, with or without compound (i.e., Compound 1) or DMSO (control). After 24 hours, tissue RNA was isolated for subsequent analysis. RNA extracts were quantified with the Qubit fluorometer (Invitrogen) and quality assessed by TapeStation automated electrophoresis (Agilent Technologies). Total RNA (250 ng) treated with amplification-grade DNase I (Invitrogen) were enriched for non-ribosomal RNA using the NEBNext rRNA Depletion Kit v2 (New England BioLabs). Library preparation was performed with the NEBNext Ultra II Directional RNA Library Prep Kit for Illumina (New England BioLabs) following the manufactures protocol for library preparation of intact or partially degraded RNA (section 2). For comparability, the same protocol was used for all samples. RNA-seq libraries were pooled and paired-end sequencing was performed on the NextSeq 2000 (Illumina) with an average sequencing depth of 200 M reads. FASTQ files were processed with Nextflow RNASeq pipeline (v. 22.10.1). Differentially expressed genes (DEG's) were identified by normalizing gene counts with DESeq2 package in R (v. 4.1.1) and applying a log2 fold change cut-off of 1.5. The effects of JAK1 inhibition with Compound 1 on DEGs identified in the prior analysis were further evaluated. In addition to the pathway analysis discussed above, individual differentially expressed genes (DEGs) in the Gimenez-Arnau and Patel studies were compared to identify a distinct CSU gene signature that was shared across both studies. Differential expression analysis was performed between lesional vs non-lesional skin, lesional vs healthy skin, and non-lesional vs healthy. Non-lesional vs healthy comparison did not produce any differentially expressed genes in either study. Based on this analysis, seven genes were found to be contributing to disease (CCL2, S100A8, SELE, PTX3, MT2A, NNMT, TNFAIP6), with four genes being associated with lesional skin (CCL2, S100A8, SELE and PTX3). Finally, the effect of JAK inhibition on cultured CSU skin to these four genes was examined. All four genes CCL2, S100A8, SELE and PTX3 showed altered expression in the presence of JAK1 inhibition. As seen in FIG. 1, PTX3 expression was dramatically reduced in CSU skin following 24 hour ex vivo treatment with JAK1 inhibition. These results indicate the effectiveness of JAK1 inhibitors, an in particular Compound 1, to treat CSU.

### Example D

### Phase 2 Study

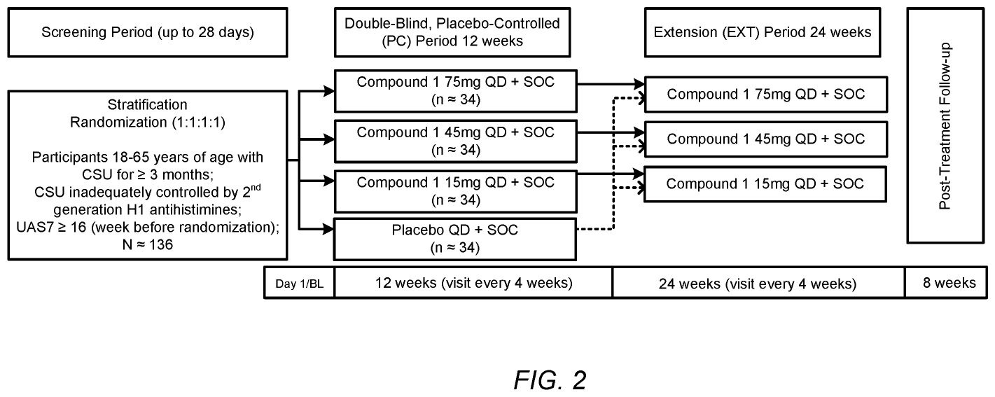

A phase 2 randomized, double-blind, placebo-controlled dose-ranging study of the efficacy and safety of JAK inhibitor Compound 1 (4-[3-(cyanomethyl)-3-(3′,5′-dimethyl-1H,l′H-4,4′-bipyrazol-1-yl)azetidin-1- yl]-2,5-difluoro-N-[(1S)-2,2,2-trifluoro-1-methylethyl]benzamide or a pharmaceutically acceptable salt thereof) for treatment of CSU (Chronic Spontaneous Urticaria) patients is described. FIG. 2 depicts an outline of a phase 2 randomized, double-blind, placebo-controlled dose-ranging study of the efficacy and safety of Compound 1. This is a Phase 2, double-blind, placebo-controlled, multicenter study of Compound 1 in participants with CSU on stable SOC with second-generation H1 antihistamines. The study will include up to 28 days for screening, continuous treatment for 36 weeks (including the PC and EXT periods), and 60 (±7) days for follow-up after the last dose of study drug. It is estimated that an individual will participate for approximately 11 months.

In some embodiments, medication will be administered orally via a tablet. Dosage will be 15 mg, 45 mg, or 75 mg of Compound 1. Dosage may be administered in the form of 1 or more 15 mg tablets. Compound 1 or matching placebo will be taken orally QD with water at approximately the same time each day unless otherwise instructed by site personnel. The study drug can be taken with or without food. Note: Dose will be administered at the study site during visits. Participants will withhold self-administration on the days of those visits. All participants will be required to remain on a concurrent, stable dose of second-generation H1 antihistamine as background therapy (SOC), which will not be changed throughout the study.

The screening period can include 28 days. The study period can include 12 weeks. The EXT period can include 24 weeks.

Inclusion criteria for participants for the study include CSU refractory to second-generation H1 antihistamines as defined by one or more of the following:

a. The presence of persistent (almost daily) itch and hives for >6 weeks at any time prior to screening despite current use of second-generation H1 antihistamines, consistent with SOC during this time period.

b. UAS7≥16 during the 7 days prior to randomization (Day 1).—Note: Participants must have at least 5 nonmissing UAS daily scores out of the 7 days before Day 1 to calculate UAS7.

c. Participants must have been on a stable dose of second-generation H1 antihistamine, consistent with SOC therapy for CSU, starting at least 3 consecutive days immediately prior to the screening visit through Day 1, and participants must agree to maintain the stable dose of second-generation H1 antihistamine throughout study and document its use.

Inclusion criteria for participants for the study include willingness to use contraception. The study can include men and women of 18 to 65 years of age with CSU for at least 3 months.

In some embodiments, criteria for participants to be excluded from the study include any one of the following: treatment with an anti-IgE biologic (e.g., omalizumab) within 8 weeks prior to screening; clearly defined predominant or sole trigger of chronic urticaria (chronic inducible urticaria) including urticaria factitial (symptomatic demographism), cold-, heat-, solar-, pressure-, delayed pressure—, aquagenic, cholinergic-, or contact-urticaria; other cutaneous or systemic diseases with symptoms of urticaria or angioedema; other skin or systemic diseases associated with chronic itching (e.g., AD, bullous pemphigoid, dermatitis herpetiformis, senile pruritus, or psoriasis); or women who are pregnant or considering pregnancy or breastfeeding. In some embodiments, criteria for participants to be excluded from the study include any one of the following concurrent conditions: thrombocytopenia, coagulopathy, or platelet dysfunction; venous and arterial thrombosis, deep vein thrombosis, pulmonary embolism, stroke, moderate to severe heart failure (NYHA Class III or IV), cerebrovascular accident, MI, coronary stenting, or CABG surgery; diagnosis of other significant cardiovascular diseases, including but not limited to angina, peripheral arterial disease, or uncontrolled arrhythmias such as atrial fibrillation, supraventricular tachycardia, ventricular tachycardia, and forms of carditis; uncontrolled hypertension, as defined by a confirmed systolic blood pressure >160 mm Hg or diastolic blood pressure >100 mm Hg; participants who are permanently bedridden or wheelchair assisted; recipient of an organ transplant that requires continued immunosuppression; any malignancies or history of malignancies with the exception of adequately treated or excised nonmetastatic basal cell or squamous cell cancer of the skin, or cervical carcinoma in situ; conditions that could interfere with drug absorption, including but not limited to short-bowel syndrome; chronic or recurrent infectious disease, including but not limited to chronic renal infection, chronic chest infection (e.g., bronchiectasis), recurrent urinary tract infection (recurrent pyelonephritis or chronic nonremitting cystitis), fungal infection, prior prosthetic joint infection at any time, or open, draining, or infected skin wounds or ulcers; current or history of disseminated herpes zoster, or recurrent (more than 1 episode of) dermatomal herpes zoster; current or history of disseminated herpes simplex; active systemic infection or any active infection that, based on the investigator's clinical assessment, makes the participant an unsuitable candidate for the study; any other active skin disease or condition (e.g., bacterial, fungal, or viral infection) that may interfere with the course, severity, or assessments of CSU; any clinically significant medical condition (other than CSU) or any other reason that the investigator determines would interfere with the participant's participation in this study or would make the participant an unsuitable candidate to receive study drug or would put the participant at risk by participating in the study; any clinically significant medical condition other than CSU, as determined by the investigator, that is not adequately controlled with appropriate treatment or may interfere with the course, severity, or assessments of CSU; or albinism.

In some embodiments, criteria for participants to be excluded from the study include a screening 12-lead ECG that demonstrates clinically significant abnormalities requiring treatment (e.g., acute MI, serious tachyarrhythmias or bradyarrhythmias) or that is indicative of serious underlying heart disease (e.g., cardiomyopathy, major congenital heart disease, low voltage in all leads, Wolff-Parkinson-White syndrome) or criteria associated with Q wave interval (QT)/Fridericia-corrected Q wave interval (QTcF) abnormalities. In some embodiments, criteria for participants to be excluded from the study include significant trauma or major surgery (per investigator's assessment) within 30 days preceding the screening visit. In some embodiments, criteria for participants to be excluded from the study include drug or alcohol abuse; treatment failure with any systemic or topical JAK inhibitor; receipt of medical treatment or investigational drugs within a predetermined time period before beginning the trial; or concurrent enrollment in another clinical study. At the screening visit, any of the laboratory abnormalities defined in Table 2.

TABLE 2
Laboratory Parameter

Exclusion Criterion

Hematology

| | | |
|---|---|---|
| a | Platelets | <100 × 10⁹/L |
| b | Hemoglobin | <10 g/dL |
| c | ANC | <1.5 × 10⁹/L |
| d | Total WBC count (leukocyte count) | <3.0 × 10⁹/L |
| e | Absolute lymphocyte count | <0.8 × 10⁹/L |

Hepatic

| | | |
|---|---|---|
| f | ALT | >2 × ULN |
| g | AST | >2 × ULN |
| h | Total bilirubin | >1.5 × ULN |
| | | (Note: Participants with clinical diagnosis of Gilbert |
| | | syndrome may have a direct bilirubin measured and |
| | | would be eligible provided the direct bilirubin is < ULN) |

Renal

| | | |
|---|---|---|
| i | Estimated glomerular filtration rate | <45 mL/min per 1.73 m² |
| | | Note: Based on the simplified, 4-variable Modification of |
| | | Diet in Renal Disease Formula |

In some embodiments, criteria for participants to be excluded from the study include any one of the following: evidence of infection with Mycobacterium tuberculosis; active HIV or acquired immunodeficiency syndrome; evidence of HBV or HCV infection or risk of reactivation; known hypersensitivity or severe reaction to Compound 1 or excipients of Compound 1; or any condition, laboratory result, or result of screening assessments that would, in the investigator's and sponsor's (or designee's) judgment, interfere with full participation in the study.

In some embodiments, efficacy of the treatment method disclosed herein can be established based upon a change from baseline in the UAS7, defined as the 7-day sum of the individual, daily recorded scores for HSS and ISS, at Week 12. Efficacy of the treatment methods disclosed herein can be established based on proportion of participants who achieve UAS7≤6 (controlled disease) at Week 12; time to first achievement of UAS7≤6 (controlled disease) during the PC period; or a proportion of participants with UAS7=0 at Week 12.

Efficacy of the treatment methods disclosed herein can be established based on a reduction of the severity of hives. Severity of hives can be evaluated based on a change from baseline in weekly HSS7 at each visit up to Week 36; a proportion of participants with HSS7=0 at Week 12; or a time to first achievement of HSS7 MID (≥5-point) improvement from baseline up to Week 36.

Efficacy of the treatment methods disclosed herein can be established based on a reduction of the severity of itch. Severity of itch can be evaluated based on a change from baseline in weekly ISS7 at each visit up to Week 36; a proportion of participants with ISS7=0 at Week 12; or a time to first achievement of ISS7 MID (≥5-point) improvement from baseline up to Week 36.

Efficacy of the treatment methods disclosed herein can be established based on a reduction of the severity of angioedema. Severity of angioedema can be evaluated based on a change from baseline in weekly AAS7 at each visit up to Week 36; a proportion of participants with AAS7=0 at Week 12; or a time to first achievement of AAS7 MID (≥5-point) improvement from baseline up to Week 36.

Efficacy of the treatment methods disclosed herein can be established based on disease control. Disease control can be evaluated based on a change from baseline in UCT7 at each visit up to Week 36; a time to first achievement of UAS7≥49.5-point improvement from baseline up to Week 36; a proportion of participants requiring treatment of CSU with corticosteroids from baseline up to Week 36; a proportion of participants requiring rescue treatment of CSU with additional second-generation H1 antihistamine from baseline up to Week 36; or a time to use of first rescue therapy from baseline up to Week 36.

Efficacy of the treatment methods disclosed herein can be established based on quality of life and other PROs. Quality of life can be evaluated based on a change from baseline in DLQI score at specified visits up to Week 36; a change from baseline in CU-Q2oL score at specified visits up to Week 36; a change from baseline in WPAI-CU score at specified visits up to Week 36; a change from baseline in AE-QoL score at specified visits up to Week 36; a change from baseline in EQ-5D-5L score at specified visits up to Week 36; a proportion of participants achieving ≥4-point decrease in DLQI score up to Week 36; a proportion of participants with a DLQI score of 0 or 1 at Week 12; a change from baseline in PGI-C at specified visits up to Week 36; or a change from baseline in PGI-S at specified visits up to Week 36.

Efficacy of the treatment methods disclosed herein can be established based on maintenance of response during the EXT period. Maintenance of response during the EXT period can be evaluated based on a proportion of participants who achieve UAS7≤6 at Week 12 and maintain or improve their response at every visit through to Week 36 or EOT2; or a proportion of participants who achieve UAS7=0 at Week 12 and maintain their response at every visit through to Week 36 or EOT2.

Efficacy of the treatment methods disclosed herein can be established based on persistence of response during the post-treatment follow-up period. Persistence of response during the post-treatment follow-up period can be evaluated based on a change from baseline in weekly HSS7 during the post-treatment follow-up period (EOT2 to EOS); a change from baseline in weekly ISS7 during the post-treatment follow-up period (EOT2 to EOS); a change from baseline in weekly AAS7 during the post-treatment follow-up period (EOT2 to EOS); a proportion of participants using rescue medication during the post-treatment follow-up period (EOT2 to EOS); or a time to use of rescue medication during the post-treatment follow-up period (EOT2 to EOS).

Efficacy of the treatment methods disclosed herein can be established based on characterizing the PK and determining systemic exposure. Characterizing the PK and determining systemic exposure can be evaluated based on a plasma concentrations of Compound 1 at specified timepoints; or a model-predicted PK exposures at steady state such as the peak, trough, and average concentrations, as well as the time to peak and terminal elimination half-life.

Efficacy of the treatment methods disclosed herein can be established based on participant heterogeneity and the effect of Compound 1 on blood biomarkers. Participant heterogeneity and the effect of Compound 1 on blood biomarkers. can be evaluated based on an expression level of biomarkers in peripheral blood before and/or after Compound 1 treatment; or a change from baseline in the expression of biomarkers in peripheral blood after Compound 1 treatment.

### Statistical Analyses

In some embodiments, a primary analysis will be primary analysis will be performed in the full analysis set (FAS). The mean change from baseline at Week 12 in weekly UAS7 score will be assessed using an mixed model (MMRM) for repeated measures to include all available data at postbaseline visits in the placebo-controlled (PC) period up to Week 12. Participants who have a baseline value and at least 1 postbaseline value in the PC period will be included in the analysis. The MMRM will include change from baseline to Week 12 as a response variable, and the fixed effect of treatment group, randomization stratification factor (previous use of an anti-IgE biologic [yes or no]), visit, treatment-by-visit interaction, and covariates of baseline value and baseline value by visit interaction. Unstructured covariance matrix will be assumed for the within-participant errors. Compound symmetry covariance matrix will be used if the model with unstructured variance covariance does not converge. The Kenward-Roger method will be used to estimate the degrees of freedom. Missing data will not be imputed. The least squares mean estimates for each treatment group and the associated covariance matrix obtained from the MMRM will be provided and further used in generalized MCP-mod framework.

In some embodiments, at the MCP stage, a contrast test statistics and multiplicity adjusted p-value for the contrast test will be provided for each prespecified candidate model. A dose-response relationship will be declared if at least 1 model among the set of 6 prespecified candidate models is identified to be statistically significant at the level a =0.025 1-sided trend test. Once a significant dose-response is established, the best model will be selected using Akaike Information Criterion.

In some embodiments, at the Mod stage, the selected model will be used to obtain the dose-response curve and the 95% CI.

In some embodiments, a secondary efficacy analysis will be primary analysis will be performed in the FAS. The odds ratios for the proportion of participants with UAS7≤6 at Week 12 in each of the Compound 1 groups and the placebo group and the corresponding 95% CIs will be assessed using logistic regression with treatment and stratification factor (previous use of an anti-IgE biologic [yes or no]). All participants who have not achieved UAS7≤6 in the PC period, as well as all participants who are missing postbaseline values, will be defined as nonresponders for the nonresponder imputation analysis.

The odds ratios for the proportion of participants with UAS7=0 at Week 12 in each of the Compound 1 groups and the placebo group and the corresponding 95% CIs will be assessed using logistic regression with treatment and stratification factor (previous use of an anti-IgE biologic [yes or no]). All participants who have not achieved UAS7=0 in the PC period, as well as all participants who are missing postbaseline values, will be defined as nonresponders for the nonresponder imputation analysis.

Time to first achievement of UAS7≤6 during the PC period is defined as the time from the date of randomization until the earliest date of achieving UAS7≤6 during the PC period. Participants who have not achieved UAS7≤6 during the PC period will be censored at the last available UAS7 measurement time. Summaries of time to first achievement of UAS7≤6 during the PC period will be assessed using the Kaplan-Meier method, and the estimated Kaplan-Meier curve will be displayed graphically.

All other secondary efficacy variables will be summarized using descriptive statistics. For categorical measurements, summary statistics will include the number and percentage of participants in each category. For continuous measurements, summary statistics will include the number of observations, mean, median, STD, minimum, and maximum. Summary statistics for continuous measures will be provided for baseline, the actual measurements at each visit, and the change and percentage change from baseline at each visit, if applicable.

In some embodiments, safety analyses will be conducted for the safety population. Adverse events will be coded by the MedDRA dictionary, and TEAEs (ie, AEs reported for the first time or worsening of a pre-existing event after first dose of study drug and until 60 days after the last dose of study drug) will be tabulated by preferred term and system organ class for all events, related events, and events of Grade 3 or higher.

In some embodiments, all exploratory efficacy variables will be summarized using descriptive statistics, if applicable. For categorical measurements, summary statistics will include the number and percentage of participants in each category. For continuous measurements, summary statistics will include the number of observations, mean, median, STD, minimum, and maximum. Summary statistics for continuous measures will be provided for baseline, the actual measurements at each visit, and the change and percentage change from baseline at each visit, if applicable.

In some embodiments, pharmacokinetic analyses will be performed for the PK-evaluable population. The Compound 1 plasma concentration data will be analyzed by a population PK modeling approach. Such data may be combined with data from other studies in the clinical development program to develop or refine population PK models, in which populations of healthy participants, participants with moderate to severe asthma, and/or participants with other diseases will be evaluated and included into the model if significant as a covariate. This model may be used to evaluate the effects of intrinsic and extrinsic covariates on the PK of Compound 1 and to determine measures of individual plasma exposures (such as steady-state peak, trough, and/or time-averaged concentrations). The population PK analysis will be reported separately.

In some embodiments, relationships between Compound 1 PK exposures and clinical responses (efficacy and safety) will be explored using an exposure-response analysis framework as deemed appropriate. For dichotomous endpoints, a generalized linear or nonlinear model with binomial link function will be used. For continuous endpoints, linear or nonlinear regressions will be used. Random effects on interindividual variability will be considered when supported by data. Clinical responses such as the primary and secondary efficacy responses and TEAEs with >10% incidence rate will be analyzed. The exposure-response analysis will be reported separately.

Various modifications of the invention, in addition to those described herein, will be apparent to those skilled in the art from the foregoing description. Such modifications are also intended to fall within the scope of the appended claims. Each reference cited in the present application, including all patent, patent applications, and publications, is incorporated herein by reference in its entirety.

What is claimed is:

1. A method of treating urticaria in a patient in need thereof, comprising administering to the patient a therapeutically effective amount of a compound which inhibits JAK1, or a pharmaceutically acceptable salt thereof, wherein the compound is:
{1 -{1-[3-Fluoro-2-(trifluoromethyl)isonicotinoyl]piperidin-4-yl-3-[4-(7H-pyrrolo[2,3-d]pyrimidin-4-yl)-1H- pyrazol-1-yl]azetidin-3 -yl} acetonitrile;
4-{3-(Cyanomethyl)-3[4-(7H-pyrrolo[2,3 -d]pyrimidin-4-yl)-1H-pyrazol-1-yl]azetidin-1-yl-N-[4-fluoro-2-(trifluoromethyl)phenyl]piperidine-1-carboxamide;
[3-[4-(7H-pyrrolo[2,3-d]pyrimidin-4-yl)-1H-pyrazol-1-yl]-1-(1-{[2-(trifluoromethyl)pyrimidin-4-yl]carbonyl}piperidin-4-yl)azetidin-3-yl]acetonitrile;
4-[3-(cyanomethyl)-3-(3′,5′-dimethyl-1H, 1′H-4,4′-bipyrazol-1-yl)azetidin-1-yl]-2,5-difluoro-N-[(1 S)-2,2,2-trifluoro-1-methylethyl]benzamide;
((2R, 5 S)-5-{2-[(1R)-1-hydroxyethyl]-1H-imidazo[4,5-d]thieno[3,2-b]pyridin-1-yl }tetrahydro-2H-pyran-2-yl)acetonitrile;
3-[1-(6-chloropyridin-2-yl)pyrrolidin-3-yl]-3-[4-(7H-pyrrolo[2,3-d]pyrimidin-4-yl)-1H-pyrazol-1-yl]propanenitrile;
3-(1-[1,3]oxazolo[5,4-b]pyridin-2-ylpyrrolidin-3-yl)-3-[4-(7H-pyrrolo[2,3-d]pyrimidin-4-yl)-1H-pyrazol-1-yl]propanenitrile;
4-[(4-{3 -cyano-2-[4-(7H-pyrrolo[2,3-d]pyrimidin-4-yl)-1H-pyrazol-1-yl]propyl}piperazin-1-yl)carbonyl]-3-fluorobenzonitrile;
4-[(4-{3-cyano-2{3-(7H-pyrrolo[2,3-d]pyrimidin-4-yl)-1H-pyrrol-1-yl]propyl} piperazin-1-yl)carbonyl]-3-fluorobenzonitrile;
[trans-1[4-(7H-pyrrolo[2,3-d]pyrimidin-4-yl)-1H-pyrazol-1-yl]-3-(4-{[2-(trifluoromethyl)pyrimidin-4-yl]carbonyl}piperazin-1-yl)cyclobutyl]acetonitrile;
{trans-3-(4-{[4-[(3-hydroxyazetidin-1-yl)methyl]-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
{trans-3-(4-{[4-{[(2S)-2-(hydroxymethyl)pyrrolidin-1-yl]methyl-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
{trans-3-{[4-{[(2R)-2-(hydroxymethyl)pyrrolidin-1-yl]methyl-6-(trifluoromethyl)pyridin-2-yl]oxy}piperidin-1-yl)-1-[4-(7H-pyrrolo[2,3-d]pyrimidin-4-y1)-1H-pyrazol-1-yl]cyclobutyl}acetonitrile;
4-(4-{[(dimethylamino)methyl]-5-fluorophenoxy}piperidin-1-yl)-3-[4-(7H-pyrrolo[2,3-d]pyrimidin-4-yl)-1H-pyrazol-1-yl]butanenitrile;
5-{3-(cyanomethyl)-3-[4-(7H-pyrrolo[2,3-d]pyrimidin-4-yl)-1H-pyrazol-1-yl]azetidin-1-yl}-N-isopropylpyrazine-2-carboxamide;
4-{3-(cyanomethyl)-3-[4-(7H-pyrrolo[2,3 -d]pyrimidin-4-yl)-1H-pyrazol-1-yl]azetidin-1-yl}2,5-difluoro-N-[(1S)-2,2,2-trifluoro-1-methylethyl]benzamide;
5-{ 3 -(cyanomethyl)-3 -[4-(1H-pyrrolo[2,3 -b]pyri din-4-yl)-1H-pyrazol-1-yl]azetidin-1-yl}-N-isopropylpyrazine-2-carboxamide;
{1-(cis-4-{[6-(2-hydroxyethyl)-2-(trifluoromethyl)pyrimidin-4-yl]oxy}cyclohexyl)-3-[4-(7H-pyrrolo[2,3-d]pyrimidin-4-yl)-1H-pyrazol-1-yl]azetidin-3-yl}acetonitrile;
{1-(cis-4-{[4-[(ethylamino)methyl]-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;
{1-(cis-4-{[4-(1-hydroxy-1-methylethyl)-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;
{1-(cis-4-{[4-{[(3R)-3-hydroxypyrrolidin-1-yl]methyl}1-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;
{1-(cis-4-{[4-{[(3S)-3-hydroxypyrrolidin-1-yl]methyl}-6-(trifluoromethyppyridin-2-yl]oxy}cyclohexyl)-3-[4-(7H-pyrrolo[2,3-d]pyrimidin-4-yl)-1H-pyrazol-1-yl]azetidin-3-yl1acetonitrile;
{trans-3-(4-{[4-({[(1S)-2-hydroxy-1-methylethyl]amino methyl)-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
{trans-3-(4-{[(2R)-2-hydroxypropyl]amino}methyl)-6-(trifluoromethyppyridin-2-yl]oxy}piperidin-1-yl)-1-[4-(7H-pyrrolo[2,3-d]pyrimidin-4-yl)-1H-pyrazol-1-yl]cyclobutyl}acetonitrile{trans-3-(4-{[4-({[(2S)-2-hydrox;
ypropyl]amino}methyl)-6-(trifluoromethyl)pyridin-2-yl]oxy}piperidin-1-yl)-1-[4-(7H-pyrrolo[2,3 -d]pyrimidin-4-yl)-1H-pyrazol-1-yl]cyclobutyl}acetonitrile;
{trans-3-(4-{[4-(2-hydroxyethyl)-6-(trifluoromethyl)pyridin-2-yl]oxy}piperidin-1-yl)-1[4-(7H-pyrrolo[2,3-d]pyrimidin-4-yl)-1H-pyrazol-1-yl]cyclobutyl}acetonitrile;
or a pharmaceutically acceptable salt of any of the aforementioned.

2. The method of claim 1, wherein the compound or salt is selective for JAK1 over JAK2, JAK3, and TYK2.

3. The method of claim 1, wherein the compound is {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

4. The method of claim 1, wherein the salt is {1-{1-[3-fluoro-2-(trifluoromethyl)isoniotinoyl]piperidin-4-yl}-3-[4-(7H-pyrrolo[2,3-d]pyrimidin-4-yl)-1H-pyrazol-1 -yl]azetidin-3-yl}acetonitrile adipic acid salt.

5. The method of claim 1, wherein the compound is 4-[3-(cyanomethyl)-3-(3′,5′-dimethyl-1H, 1H,1H′-4,4′-bipyrazol-1-yl)azetidin-1-yl]-2,5-difluoro-N-[(1S)-2,2,2-trifluoro-1-methylethyl]benzamide, or a pharmaceutically acceptable salt thereof.

6. The method of claim 1, wherein the salt is 4-[3-(cyanomethyl)-3-(3′,5′-dimethyl-1H,1′H-4,4′-bipyrazol-1-yl)azetidin-1- yl]-2,5-difluoro-N-[(1S)-2,2,2-trifluoro-1-methylethyl]benzamide phosphoric acid salt.

7. The method of claim 1, wherein the compound is ((2R,5S)-5-{2-[(1R)-1-hydroxyethyl]1H-imidazo[4,5 -d]thieno[3,2-b]pyridin-1-yl}tetrahydro-2H-pyran-2-yl)acetonitrile, or a pharmaceutically acceptable salt thereof.

8. The method of claim 1, wherein the compound is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile monohydrate.

9. The method of claim 1, wherein the compound or salt is administered at a dosage of 15, 30, 45, or 75 mg on a free base basis.

10. The method of claim 1, wherein the compound or salt is administered at a dosage of 15, 45, or 75 mg on a free base basis.

11. The method of claim 1, wherein the compound or salt is administered at a dosage of 15 mg or 45 mg on a free base basis.

12. The method of claim 1, further comprising administering an additional therapeutic agent.

13. The method of claim 12, wherein the additional therapeutic agent is an antihistamine.

14. The method of claim 13, wherein the antihistamine is a second-generation H1 antihistamine.

15. The method of claim 12, wherein the additional therapeutic agent is an antibiotic, a retinoid, a corticosteroid, an anti-TNF-alpha agent, or an immunosuppressant.

16. The method of claim 15, wherein the antibiotic is clindamycin, doxycycline, minocycline, trimethoprim-sulfamethoxazole, erythromycin, metronidazole, rifampin, moxifloxacin, dapsone, or a combination thereof.

17. The method of claim 15, wherein the retinoid is etretinate, acitretin, or isotretinoin.

18. The method of claim 15, wherein the corticosteroid is triamcinolone, dexamethasone, fluocinolone, cortisone, prednisone, prednisolone or flumetholone.

19. The method of claim 15, wherein the anti-TNF-alpha agent is infliximab, etanercept, or adalimumab.

20. The method of claim 15, wherein the immunosuppressant is methotrexate, cyclosporin A, mycophenolate mofetil, or mycophenolate sodium.

21. The method of claim 15, wherein the additional therapeutic agent is finasteride, metformin, adapalene, or azelaic acid.

22. The method of claim 1, wherein the administrating of the compound or salt is topical.

23. The method of claim 1, wherein the administering of the compound or salt is oral.

24. The method of claim 1, wherein the methods results in about a 10% to about a 90% improvement in a number and/or size of welts.
